(19) United States
(12) Patent Application Publication
(10) Pub. No.: US 2026/0290052 A1
(43) Pub. Date: Sep. 24, 2026

(54) SYSTEM AND METHOD FOR CLASSIFICATION OF BASAL CELL CARCINOMA BASED ON CONFOCAL MICROSCOPY

(71) Applicants: National Skin Centre (Singapore) PTE Ltd, Singapore (SG); Nanyang Technological University, Singapore (SG); Agency for Science, Technology and Research, Singapore (SG)

(72) Inventors: Steven Tien Guan THNG, Singapore (SG); Sai Yee CHUAH, Singapore (SG); Jun XIE, Singapore (SG); Wai-Kin Adams KONG, Singapore (SG); Li LIN, Singapore (SG)

(21) Appl. No.: 18/874,410

(22) PCT Filed: May 29, 2023
(86) PCT No.: PCT/SG2023/050380

§ 371 Date: Dec. 12, 2024

(30) Foreign Application Priority Data
Jun. 24, 2022 (SG) 10202250290Q

(51) Int. Cl. G06V 20/69; G06V 10/26; G06V 10/764; G06V 10/774; G16H 50/20; G16H 50/70

(52) U.S. Cl. CPC G06V 20/698; G06V 10/26; G06V 10/764; G06V 10/774; G06V 20/695; G16H 50/20; G16H 50/70; G06V 2201/03

(57) ABSTRACT

A system for classification of basal cell carcinoma (BCC) and a method of classifying basal cell carcinoma (BCC) may be provided, the system comprising a processing module configured to receive an input image sequence comprising a plurality of images; and a prediction module coupled to the processing module, the prediction module comprising a hierarchical ensemble structure of a plurality of classification models; wherein the processing module is configured to process each image as a whole image to obtain a plurality of first sub-sections and to further process each first sub-section to obtain a corresponding plurality of second sub-sections; further wherein the processing module is configured to transmit the each first sub-section to a first classification model of the prediction module trained to process the first sub-sections and to transmit each of the corresponding plurality of second sub-sections to a second classification model of the prediction module trained to process the second sub-sections, further wherein the prediction module is configured to determine a probability of BCC of the each image of the input image sequence based on a plurality of respective block prediction results corresponding to the first sub-sections of the each image; and further wherein the prediction module is configured to determine a BCC classification of the input image sequence based on the determined probability of BCC of each image of the input image sequence.

### TECHNICAL FIELD

The present disclosure relates broadly to a system for classification of basal cell carcinoma based on confocal microscopy and to a method of classifying basal cell carcinoma using confocal microscopy.

### BACKGROUND

Basal-cell carcinoma (BCC) is typically recognised as the most common type of skin cancer. It currently constitutes more than 80% of non-melanoma skin cancer cases, and 32% of all skin cancer cases globally. Compared with other types of skin cancer, BCC has a lower mortality and morbidity rate due to its slow progression and low metastasis potential. However, it has been reported that there is a lifetime risk of 30% of developing a BCC. Furthermore, BCC can cause a significant social healthcare burden, as well as increase individual disability-life adjusted years. It was reported that in 2017, non-melanoma skin cancer, of which BCC constitutes a majority of the cases, caused about 65000 deaths and about 1.3 million disability-life adjusted years. Due to the relatively low mortality rate for example, however, both individuals and healthcare systems may not be paying sufficient attention to BCC. Researchers have pointed out that the number of BCC cases is under-reported, and most data comes from developed countries like Canada, the United States, Australia and the United Kingdom, while Africa has the lowest incident rate as well as limited available data. As such, in view of the various risks above, there exists a need to enhance diagnosis of BCC.

Clinical diagnosis of BCC currently relies on two methods, dermoscopy and histopathology. Dermoscopy is a widely used method in BCC diagnosis. It is non-invasive and is a relatively cheap method. It can typically provide 10~50× magnification of superficial skin for physicians to scrutinise lesions. Histopathology on the other hand is currently the gold standard used in BCC diagnosis, based on dyed lesion biopsies, which are typically biopsied for showing suspicious lesions. Other imaging methods such as in vivo confocal microscopy, ultrasonography and optical coherence tomography (OCT) have also shown promising potential in BCC detection.

It is recognised that in the above-mentioned methods, the final diagnostic decisions are typically still manually made by a human healthcare professional recognising certain patterns of BCC, which is time-consuming and costly. Furthermore, for manual classification of confocal BCC images, human healthcare professionals (e.g., dermatologists) mostly rely on features such as tumour islands, peripheral palisadings, cleftings, as well as vascularity around tumour islands. However, it is recognised that not all BCC cases may demonstrate every feature mentioned above, and it is recognised that practically, many BCC features (e.g., BCC lesions) may not be observable within each image of a scanning sequence. Therefore, it is recognised that it may be easy to miss BCC lesions in scanning results, especially if performed by untrained personnel. As such, in view of the practical limitations above, there exists a need to assist or facilitate diagnosis of BCC.

Further, as the gold standard for BCC diagnosis, histopathology is based on invasive biopsy sampling of a lesion. Therefore, histopathology is often used as a means for final determination and guidance before radical treatment, instead of as a means for early detection. On the other hand, dermoscopy can typically only provide superficial information of the skin, which may not provide comprehensive information of a lesion. Due to the above differences between dermoscopy and histopathology, confocal microscopy is being explored as a suitable alternative. As a non-invasive medical imaging method, confocal microscopy (e.g., reflectance confocal microscopy (RCM)) can provide a sequence of scanning images each taken at a different depth beneath the skin surface. Confocal microscopy can also be favourable in that it has an optical resolution comparable with the typical resolution used for histopathology. Further, as a non-invasive method, confocal microscopy can make extensive and regular examination of BCC possible, i.e., providing more information than dermoscopy and providing a non-invasive alternative to histopathology. Upon a comparison of spatial resolution and penetration depth of different medical imaging methods such as Optical Coherence Tomography (OCT) and ultrasonography, the inventors recognise that confocal microscopy can provide higher spatial resolution than such methods, and that confocal microscopy can provide more morphological information of BCC lesions than such methods.

However, it has been recognised by the inventors that even with confocal microscopy, there still exists the problem of having to rely on the expertise of a human healthcare professional or trained personnel (e.g., an experienced dermatologist for clinical diagnosis), which incurs costs (e.g., due to a need for training) and which may be inefficient (e.g., due to a lack of such trained personnel that may lead to potential backlogs for diagnosis cases).

In view of the above, there exists a need to address or at least ameliorate the above-mentioned problems. In particular, there exists a need to provide a system for classification of basal cell carcinoma (BCC) based on confocal microscopy and a method of classifying basal cell carcinoma using confocal microscopy that address or at least ameliorate one or more of the above-mentioned problems.

### SUMMARY

In accordance with an aspect of the present disclosure, there is provided a system for classification of basal cell carcinoma (BCC), the system comprising a processing module configured to receive an input image sequence comprising a plurality of images; and a prediction module coupled to the processing module, the prediction module comprising a hierarchical ensemble structure of a plurality of classification models; wherein the processing module is configured to process each image as a whole image to obtain a plurality of first sub-sections and to further process each first sub-section to obtain a corresponding plurality of second sub-sections; further wherein the processing module is configured to transmit the each first sub-section to a first classification model of the prediction module trained to process the first sub-sections and to transmit each of the corresponding plurality of second sub-sections to a second classification model of the prediction module trained to process the second sub-sections, the processing module also configured to transmit the whole image to a third classification model of the prediction module trained to process the whole image; wherein the prediction module is configured to provide a respective block prediction result corresponding to the each first sub-section, the respective block prediction result based on respective prediction results of the first classification model for the each first sub-section and the second classification model for the corresponding plurality of second sub-sections; wherein the prediction module is configured to provide a preliminary whole image prediction result for the each image based on the third classification model; further wherein the prediction module is configured to determine a probability of BCC of the each image of the input image sequence based on the preliminary whole image prediction result and a plurality of the respective block prediction results corresponding to the first sub-sections of the each image; and further wherein the prediction module is configured to determine a BCC classification of the input image sequence based on the determined probability of BCC of each image of the input image sequence.

The system may further comprise the prediction module being configured to determine the BCC classification of the input image sequence based on the determined probability of BCC of each image of the input image sequence along a depth of the input image sequence.

A graphical representation of the determined probability of BCC of each image of the input image sequence along the depth of the input image sequence may be generated.

The BCC classification of the input image sequence may be determined based on an equation:

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$

where Sₐᵢ refers to an area of each consecutive part of the graphical representation between a determined probability value of 0.8 to 1, S_{bj} refers to an area of each consecutive part of the graphical representation between a determined probability value of 0.6 to 0.8, S_{ck} refers to an area of each consecutive part of the graphical representation between a determined probability value of 0.4 to 0.6, lₐᵢ refers to a corresponding length of Sₐᵢ in the graphical representation, l_{bj} refers to a corresponding length of S_{bj} in the graphical representation and l_{ck} refers to a corresponding length of S_{ck} in the graphical representation.

The system may further comprise the prediction module being configured to provide the respective block prediction result corresponding to the each first sub-section by processing the respective prediction results of the first classification model for the each first sub-section and the second classification model for the corresponding plurality of second sub-sections with a block support vector machine (SVM).

The system may further comprise the prediction module being configured to determine the probability of BCC of the each image of the input image sequence by processing the preliminary whole image prediction result and the plurality of the respective block prediction results corresponding to the first sub-sections of the each image with an image-wise stage support vector machine (SVM).

The system may further comprise the processing module being configured to perform training of the first classification model, the second classification model and the third classification model using at least two training groups of images.

The at least two training groups of images may comprise a BCC training group of images and a non-BCC training group of images, the BCC training group of images comprising images of BCC subjects arranged as one or more BCC image sequences and the non-BCC training group of images comprising images of normal subjects arranged as one or more non-BCC image sequences.

The system may further comprise the processing module being configured to perform data augmentation of the BCC training group of images and the non-BCC training group of images based on a threshold value of an image average signal intensity.

The system may further comprise the processing module being configured to generate a visual heatmap based on the respective prediction results of the first classification model for the each first sub-section and the second classification model for the corresponding plurality of second sub-sections, the heatmap being generated by processing the respective prediction results of the first classification model for the each first sub-section and the second classification model for the corresponding plurality of second sub-sections to form a heatmap mask for a combination with the whole image and by providing colour transformation to the combination with the whole image.

The system may further comprise the processing module being configured to split the training groups of images into a training set, a validation set, and a test set at a ratio of 8:1:1.

The at least two training groups of images may be used for training the third classification model and are denoted as a whole image dataset.

Based on the whole image dataset, the processing module may be configured to process each image of the whole image dataset to obtain a plurality of first training sub-sections and the first training sub-sections are denoted as a first training sub-sections dataset, and wherein the first training sub-sections dataset is used for training the first classification model.

Based on the first training sub-sections dataset, the processing module may be configured to process each first training sub-sections to obtain a plurality of second training sub-sections and the second training sub-sections are denoted as a second training sub-sections dataset, and wherein the second training sub-sections dataset is used for training the second classification model.

In accordance with another aspect of the present disclosure, there is provided a method of classifying basal cell carcinoma (BCC), the method comprising providing a processing module; inputting an input image sequence comprising a plurality of images to the processing module; coupling a prediction module to the processing module, the prediction module comprising a hierarchical ensemble structure of a plurality of classification models; processing each image, by the processing module, as a whole image to obtain a plurality of first sub-sections and further processing, by the processing module, each first sub-section to obtain a corresponding plurality of second sub-sections; transmitting, by the processing module, the each first sub-section to a first classification model of the prediction module trained to process the first sub-sections; transmitting, by the processing module, each of the corresponding plurality of second sub-sections to a second classification model of the prediction module trained to process the second sub-sections; transmitting, by the processing module, the whole image to a third classification model of the prediction module trained to process the whole image; providing, by the prediction module, a respective block prediction result corresponding to the each first sub-section, the respective block prediction result based on respective prediction results of the first classification model for the each first sub-section and the second classification model for the corresponding plurality of second sub-sections; providing, by the prediction module, a preliminary whole image prediction result for the each image based on the third classification model; determining, by the prediction module, a probability of BCC of the each image of the input image sequence based on the preliminary whole image prediction result and a plurality of the respective block prediction results corresponding to the first sub-sections of the each image; and determining, by the prediction module, a BCC classification of the input image sequence based on the determined probability of BCC of each image of the input image sequence.

The method may further comprise determining, by the prediction module, the BCC classification of the input image sequence based on the determined probability of BCC of each image of the input image sequence along a depth of the input image sequence.

The method may further comprise generating a graphical representation of the determined probability of BCC of each image of the input image sequence along the depth of the input image sequence.

The BCC classification of the input image sequence may be determined based on an equation:

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$

where Sₐᵢ refers to an area of each consecutive part of the graphical representation between a determined probability value of 0.8 to 1, S_{bj} refers to an area of each consecutive part of the graphical representation between a determined probability value of 0.6 to 0.8, S_{ck} refers to an area of each consecutive part of the graphical representation between a determined probability value of 0.4 to 0.6, lₐᵢ refers to a corresponding length of Sₐᵢ in the graphical representation, l_{bj} refers to a corresponding length of S_{bj} in the graphical representation and l_{ck} refers to a corresponding length of S_{ck} in the graphical representation.

The method may further comprise providing, by the prediction module, the respective block prediction result corresponding to the each first sub-section by processing the respective prediction results of the first classification model for the each first sub-section and the second classification model for the corresponding plurality of second sub-sections with using a block support vector machine (SVM).

The method may further comprise determining, by the prediction module, the probability of BCC of the each image of the input image sequence by processing the preliminary whole image prediction result and the plurality of the respective block prediction results corresponding to the first sub-sections of the each image with using an image-wise stage support vector machine (SVM).

The method may further comprise performing, by the processing module, training of the first classification model, the second classification model and the third classification model using at least two training groups of images.

The at least two training groups of images may comprise a BCC training group of images and a non-BCC training group of images, the BCC training group of images comprising images of BCC subjects arranged as one or more BCC image sequences and non-BCC training group of images comprising images or normal subjects arranged as one or more non-BCC image sequences.

The method may further comprise performing, by the processing module, data augmentation of the BCC training group of images and the non-BCC training group of images based on a threshold value of an image average signal intensity

The method may further comprise forming a heatmap mask by processing the respective prediction results of the first classification model for the each first sub-section and the second classification model for the corresponding plurality of second sub-sections; combining the heatmap mask with the whole image; and providing colour transformation to the combination with the whole image to generate a visual heatmap.

The method may further comprise splitting, by the processing module, the training groups of images into a training set, a validation set, and a test set at a ratio of 8:1:1.

The at least two training groups of images may be used for training the third classification model and are denoted as a whole image dataset.

The method may further comprise based on the whole image dataset, processing, by the processing module, each image of the whole image dataset to obtain a plurality of first training sub-sections and the first training sub-sections are denoted as a first training sub-sections dataset, and training the first classification model with the first training sub-sections dataset.

The method may further comprise based on the first training sub-sections dataset, processing, by the processing module, each first training sub-sections to obtain a plurality of second training sub-sections and the second training sub-sections are denoted as a second training sub-sections dataset, and training the second classification model with the second training sub-sections dataset.

In accordance with another aspect of the present disclosure, there is provided a non-transitory tangible computer readable storage medium having stored thereon software instructions that, when executed by a processing module of a system for classification of basal cell carcinoma (BCC), cause the processing module to perform a method of classifying basal cell carcinoma (BCC), by executing the steps comprising, providing a processing module; inputting an input image sequence comprising a plurality of images to the processing module; coupling a prediction module to the processing module, the prediction module comprising a hierarchical ensemble structure of a plurality of classification models; processing each image, by the processing module, as a whole image to obtain a plurality of first sub-sections and further processing, by the processing module, each first sub-section to obtain a corresponding plurality of second sub-sections; transmitting, by the processing module, the each first sub-section to a first classification model of the prediction module trained to process the first sub-sections; transmitting, by the processing module, each of the corresponding plurality of second sub-sections to a second classification model of the prediction module trained to process the second sub-sections; transmitting, by the processing module, the whole image to a third classification model of the prediction module trained to process the whole image; providing, by the prediction module, a respective block prediction result corresponding to the each first sub-section, the respective block prediction result based on respective prediction results of the first classification model for the each first sub-section and the second classification model for the corresponding plurality of second sub-sections; providing, by the prediction module, a preliminary whole image prediction result for the each image based on the third classification model; determining, by the prediction module, a probability of BCC of the each image of the input image sequence based on the preliminary whole image prediction result and a plurality of the respective block prediction results corresponding to the first sub-sections of the each image; and determining, by the prediction module, a BCC classification of the input image sequence based on the determined probability of BCC of each image of the input image sequence.

### BRIEF DESCRIPTION OF THE DRAWINGS

Exemplary embodiments of the present disclosure will be better understood and readily apparent to one of ordinary skill in the art from the following written description, by way of example only, and in conjunction with the drawings, in which:

FIG. 1 is a schematic diagram of a system for classification of Basal Cell Carcinoma (BCC) in an exemplary embodiment.

FIG. 2 is a schematic diagram of a prediction module in an exemplary embodiment.

FIG. 3 is a schematic framework for generating a probability of BCC for each image of an image sequence in an exemplary embodiment.

FIG. 4A is a graph showing exemplary image-wise BCC prediction scores for a plurality of images from a RCM (reflectance confocal microscopy) input image sequence along a depth of the RCM input image sequence.

FIG. 4B is a graph illustrating data for calculating a sequence-wise BCC prediction score in an exemplary embodiment.

FIG. 5A is a schematic flowchart illustrating a method of training models in an exemplary embodiment.

FIG. 5B is an exemplary set of images below and above a pre-determined threshold signal (T).

FIG. 5C shows examples of BCC features that can be used for classification of BCC under confocal microscopy scanning.

FIG. 5D is an exemplary illustration showing training of support vector machines (SVM) models in an exemplary embodiment.

FIG. 6 is a set of heatmaps generated by a self-embedded attribution map in an exemplary embodiment.

FIG. 7 is a schematic diagram of a system framework for classification of BCC in another exemplary embodiment.

FIG. 8 is a schematic illustration of an example of generating a probability of BCC for an image of an input image sequence in another exemplary embodiment.

FIG. 9 is an example probability curve of an input image sequence in an exemplary embodiment.

FIG. 10 is a schematic flow diagram for illustrating generation of an example visual heatmap in an exemplary embodiment.

FIG. 11 is a schematic flowchart for illustrating a method of classifying BCC in an exemplary embodiment.

FIG. 12 is a schematic drawing of a computer system suitable for implementing an exemplary embodiment.

### DETAILED DESCRIPTION

It will be appreciated that where priority is claimed to an earlier application, the full contents of the earlier application is also taken to form part of the present description and those full contents provide support for embodiments disclosed herein.

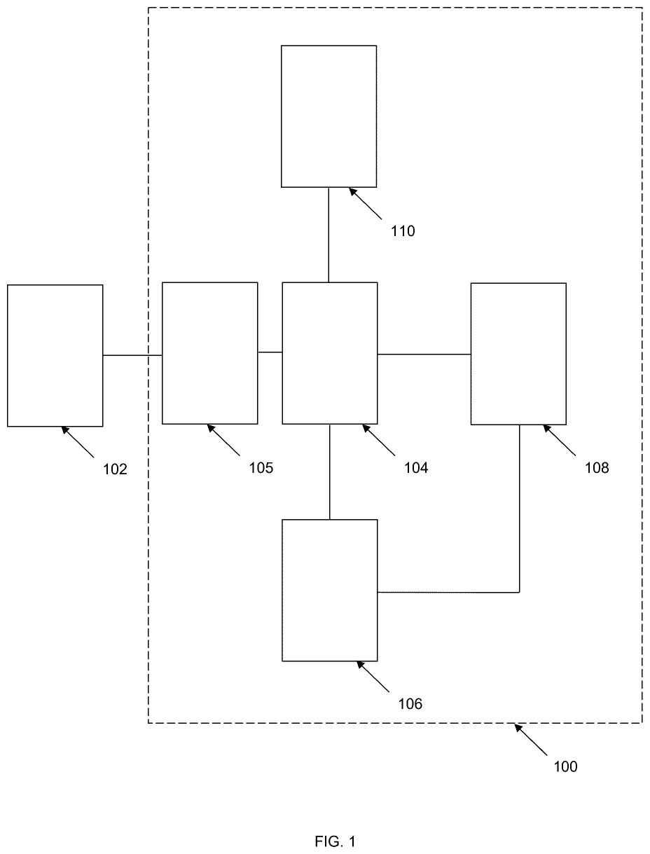

FIG. 1 is a schematic diagram of a system for classification of Basal Cell Carcinoma (BCC) in an exemplary embodiment. The system for classification of BCC is based on confocal microscopy, e.g., reflectance confocal microscopy (RCM).

In the exemplary embodiment, the system 100 may be coupled to a confocal microscopy device 102. The confocal microscopy device 102 is configured to obtain/capture and compile an input image sequence comprising a plurality of images. In the description herein, for an image sequence, each image is an image of a same skin area of a subject obtained at a pre-determined depth (and/or a consecutive depth) with respect to a skin surface of a subject. For example, each image of the image sequence may be obtained at an increasing consecutive depth with respect to the skin surface of a subject. The input image sequence therefore comprises a plurality of images of a same skin area with each image obtained by the confocal microscopy device 102 at a different pre-determined depth with respect to the skin surface of a subject. The subject, for example, may be a human subject.

In the exemplary embodiment, the system 100 comprises a processing module 104 configured to receive the input image sequence comprising the plurality of images. The processing module 104 may receive the input image sequence after some time passes from when the confocal microscopy device 102 obtains/captures and compiles the input image sequence. In the exemplary embodiment, the input image sequence obtained/captured and compiled at the confocal microscopy device 102 may be stored in a storage device (not shown), and the processing module 104 may be configured to receive the input image sequence via the storage device. The storage device may be for example, but not limited to, a removable storage device (e.g., a USB drive, an external hard drive) and/or a cloud storage device (e.g. the data being stored or transmitted over the internet). Thus, the processing module 104 may be coupled to an input member 105 that can receive the input image sequence and that is arranged to input the input image sequence comprising the plurality of images to the processing module 104.

In the exemplary embodiment, the system 100 further comprises a prediction module 106 coupled to the processing module 104. The prediction module 106 comprises a hierarchical ensemble structure of a plurality of classification models. In the exemplary embodiment, the hierarchical ensemble structure includes a first classification model, a second classification model, and a third classification model. In the exemplary embodiment, the operations of the prediction module 106 are instructed by the processing module 104.

In the exemplary embodiment, the system 100 further comprises a storage medium 110 coupled to the processing module 104. The storage medium 110 may store instructions/code that are executable by the processing module 104. In some exemplary embodiments, the processing module 104 is configured to retrieve and execute the instructions from the storage medium 110, and to provide the prediction module 106 (and/or the functions of the prediction module 106).

In the exemplary embodiment, the storage medium 110 may further comprise a deep learning database (not shown). The deep learning database stores the plurality of classification models (e.g., a first classification model, a second classification model, and a third classification model) for the hierarchical ensemble structure of the prediction module 106 e.g., for BCC classification. In the exemplary embodiment, the processing module 104 is configured to retrieve the plurality of classification models from the deep learning database for assembling the hierarchical ensemble structure.

In the exemplary embodiment, the processing module 104 is configured to process each image (of an input image sequence) as a whole image to obtain a plurality of first sub-sections and to further process each first sub-section to obtain a corresponding plurality of second sub-sections.

In the exemplary embodiment, the processing module 104 is configured to transmit each first sub-section to a first classification model of the prediction module 106 trained to process the first sub-sections and to transmit each of the corresponding plurality of second sub-sections to a second classification model of the prediction module 106 trained to process the second sub-sections. The processing module 104 is also configured to transmit the whole image to a third classification model of the prediction module 106 trained to process the whole image.

In the exemplary embodiment, the prediction module 106 is configured to provide a respective block prediction result corresponding to each first sub-section. The respective block prediction result is based on respective prediction results of the first classification model for each first sub-section and the second classification model for the corresponding plurality of second sub-sections.

In the exemplary embodiment, the prediction module 106 is configured to provide a preliminary whole image prediction result for each image based on the third classification model.

In the exemplary embodiment, the prediction module 106 is configured to determine a probability of BCC of each image of the input image sequence based on the preliminary whole image prediction result and a plurality of the respective block prediction results corresponding to the first sub-sections of each image.

In the exemplary embodiment, the probability of BCC for each image may provide an indication as to the extent to which each image (of the input image sequence) shows BCC features (e.g., BCC lesions).

In the exemplary embodiment, the prediction module 106 is configured to determine a BCC classification of the input image sequence based on the determined probability of BCC of each image of the input image sequence.

In the exemplary embodiment, the system 100 further comprises an output device 108 coupled to the processing module 104 and the prediction module 106. In the exemplary embodiment, the processing module 104 is configured to transmit the determined BCC classification of the input image sequence (determined by the prediction module 106) to the output device 108. In the exemplary embodiment, the output device 108 is configured to provide/present the determined BCC classification of the input image sequence to a user. In some exemplary embodiments, the output device 108 may be provided in the form of a monitor or a display that displays a user interface (e.g., a graphical user interface).

In the exemplary embodiment, the processing module 104 is configured to generate a visual heatmap based on the respective prediction results of the first classification model for the each first sub-section and the second classification model for the corresponding plurality of second sub-sections, the heatmap being generated by processing the respective prediction results of the first classification model for the each first sub-section and the second classification model for the corresponding plurality of second sub-sections to form a heatmap mask for a combination with the whole image and by providing colour transformation to the combination with the whole image.

In use, the confocal microscopy device 102 obtains/captures a plurality of images. Thus, there is obtained a plurality of images of a skin area of a subject, each image being at a different pre-determined depth with respect to a skin surface of the subject. The confocal microscopy device 102 compiles an input image sequence comprising the plurality of images. The input image sequences obtained/captured and compiled by the confocal microscopy device 102 is transmitted to (and received by) the processing module 104. The processing module 104 processes each image as a whole image to obtain a plurality of first sub-sections and further processes each first sub-section to obtain a corresponding plurality of second sub-sections. The processing module 104 transmits each first sub-section to a first classification model of the prediction module 106 trained to process the first sub-sections and transmits each of the corresponding plurality of second sub-sections to a second classification model of the prediction module 106 trained to process the second sub-sections. The processing module 104 also transmits the whole image to a third classification model of the prediction module 106 trained to process the whole image. The prediction module 106 provides a respective block prediction result corresponding to each first sub-section based on the respective prediction results of the first classification model for each first sub-section and the second classification model for the corresponding plurality of second sub-sections. The prediction module 106 provides a preliminary whole image prediction result for each image based on the third classification model. The prediction module 106 determines a probability of BCC of each image of the input image sequence based on the preliminary whole image prediction result and a plurality of the respective block prediction results corresponding to the first sub-sections of each image. The prediction module 106 determines a BCC classification of the input image sequence based on the determined probability of BCC of each image of the input image sequence. The processing module 104 then transmits the determined BCC classification of the input image sequence to the output device 108. The output device 108 provides/presents the determined BCC classification of the input image sequence to a user.

In the exemplary embodiment, therefore, the system 100 is configured to output the determined BCC classification of the skin area of the subject based on the input image sequence obtained and compiled by the confocal microscopy device 102, and further based on the plurality of classification models of the hierarchical ensemble structure for BCC classification. Therefore, the system 100 makes BCC detection based on confocal microscopy and deep learning or machine learning possible.

In the exemplary embodiment, the prediction module 106 is configured to determine the BCC classification of the input image sequence based on the determined probability of BCC of each image of the input image sequence along a depth of the input image sequence. A graphical representation of the determined probability of BCC of each image of the input image sequence along the depth of the input image sequence can be generated. In the exemplary embodiment, the BCC classification of the input image sequence is determined based on an equation:

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$

where Sₐᵢ refers to an area of each consecutive part of the graphical representation between a determined probability value of 0.8 to 1, S_{bj} refers to an area of each consecutive part of the graphical representation between a determined probability value of 0.6 to 0.8, S_{ck} refers to an area of each consecutive part of the graphical representation between a determined probability value of 0.4 to 0.6, lₐᵢ refers to a corresponding length of Sₐᵢ in the graphical representation, l_{bj} refers to a corresponding length of S_{bj} in the graphical representation and l_{ck} refers to a corresponding length of S_{ck} in the graphical representation.

In the exemplary embodiment, in use, the prediction module 106 provides the respective block prediction result corresponding to each first sub-section by processing the respective prediction results of the first classification model for each first sub-section and the second classification model for the corresponding plurality of second sub-sections with a block support vector machine (SVM).

In the exemplary embodiment, in use, the prediction module 106 determines the probability of BCC of each image of the input image sequence by processing the preliminary whole image prediction result and the plurality of the respective block prediction results corresponding to the first sub-sections of each image with an image-wise stage support vector machine (SVM).

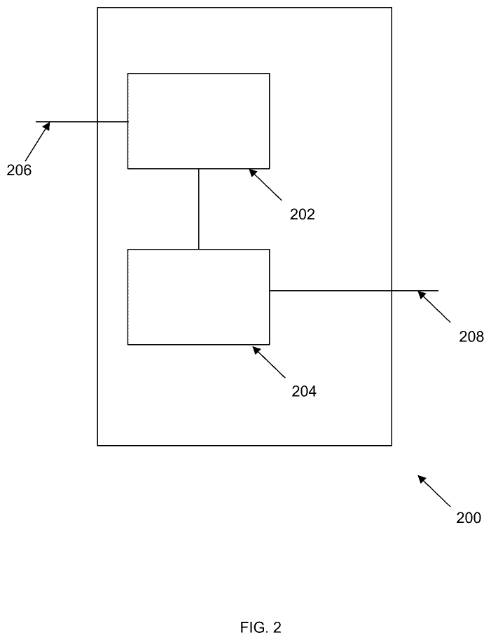

FIG. 2 is a schematic diagram of a prediction module in an exemplary embodiment. In this exemplary embodiment, the prediction module 200 functions substantially similarly to the prediction module 106 described with reference to FIG. 1. The operations of the prediction module 200 may be instructed by a processing module (e.g., see processing module 104 described with reference to FIG. 1).

In the exemplary embodiment, the prediction module 200 comprises an image-wise prediction module 202 and a sequence-wise prediction module 204. The prediction module 200 comprises an input 206 and an output 208.

In the exemplary embodiment, the image-wise prediction module 202 is configured to receive, at the input 206, a plurality of images of an input image sequence (e.g., the plurality of images of the input images sequences obtained/captured by the confocal microscopy device 102 described with reference to FIG. 1). The images may have been obtained at consecutive depths with respect to a skin surface of a subject.

The image-wise prediction module 202 is configured to then process the plurality of images individually and to output a probability of BCC (or an image-wise BCC prediction score) for each image of the input image sequence. In the exemplary embodiment, the probability of BCC for each image may provide an indication as to the extent to which each image (of the input image sequence) shows BCC features (e.g., BCC lesions). In one exemplary embodiment, each probability of BCC is in the form of a value in a range between 0 (zero) to 1 (one), both inclusive, with 1 indicating that the probability of the image (of the input image sequence) containing BCC features is 100%, and 0 indicating that the probability of the image (of the input image sequence) containing BCC features is 0%.

In the exemplary embodiment, the sequence-wise prediction module 204 is configured to receive the determined probability of BCC of each image of the input image sequence from the image-wise prediction module 202. The sequence-wise prediction module 204 is configured to process the determined probabilities of BCC, and to output a BCC classification of the input image sequence at the output 208. In one exemplary embodiment, the BCC classification may be in the form of a value which can be used to evaluate whether the subject (from whom the input image sequence was obtained) may have BCC.

In the exemplary embodiment, in use, the prediction module 200 implements a two-stage method of generating a BCC classification for an input image sequence. In the exemplary embodiment, the two stages are, namely, an image-wise BCC prediction stage and a sequence-wise BCC prediction stage. In the image-wise BCC prediction stage, a probability of BCC is generated/determined for each image of the input image sequence by the image-wise prediction module 202. In the sequence-wise prediction stage, a BCC classification is generated for the entire input image sequence by the sequence-wise prediction module 204, based on the probability of BCC generated for each image of the input image sequence by the image-wise prediction module 202.

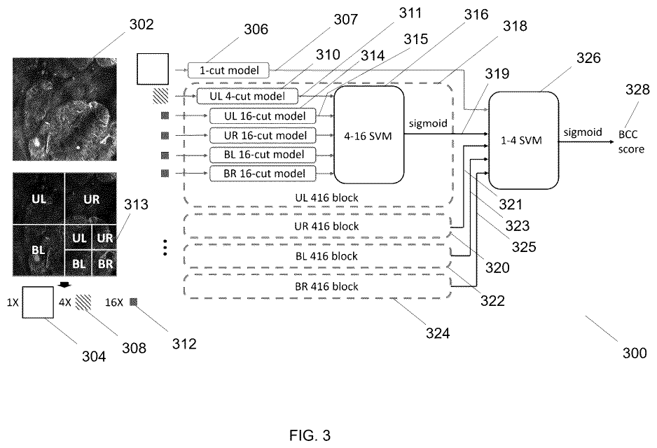

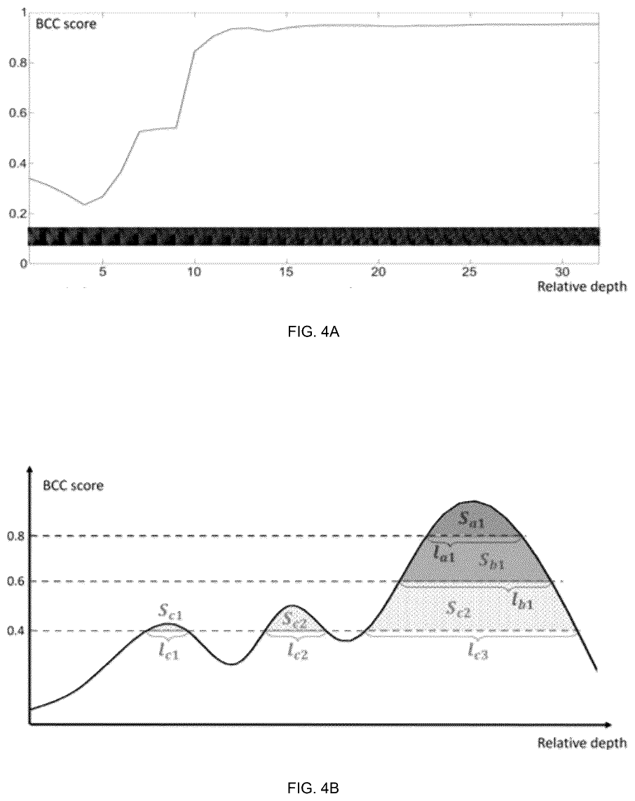

An exemplary framework for generating a probability of BCC for each image of the input image sequence by the image-wise prediction module 202 is described with reference to FIG. 3. An exemplary method of generating a BCC classification by the sequence-wise prediction module 204 is described with reference to FIGS. 4A and 4B.

FIG. 3 is a schematic framework for generating a probability of BCC for each image of an image sequence in an exemplary embodiment. In the exemplary embodiment, the framework 300 may be implemented/performed by a processing module (e.g., see processing module 104 described with reference to FIG. 1) with the use of an image-wise prediction module of a prediction module (e.g., see image-wise prediction module 202 of prediction module 200 described with reference to FIG. 2). For ease of reference, like naming conventions are used for exemplary implementations of similar modules as described in FIG. 1 and FIG. 2.

In the exemplary embodiment, a reflectance confocal microscopy (RCM) device may be used to obtain/capture and compile an input image sequence comprising a plurality of RCM images. A typical size of a RCM image may be 1000×1000 pixels, and a typical input size for an image classification model may be 224×224 pixels. It has been recognised by the inventors that a simple re-sizing of images may cause a significant information loss of more than 95%. Thus, in the framework 300, for generating a probability of BCC (referred as an image-wise BCC prediction score in the exemplary embodiment described with reference to FIG. 3) for each image of an input image sequence, to utilise the high spatial resolution that can be provided by RCM without losing the global features, a hierarchical ensemble scheme/structure of a plurality of classification models is used.

In the exemplary embodiment, a plurality of RCM images of an input image sequence, each image being 1000×1000 pixels, is transmitted to an image-wise prediction module of a prediction module (e.g., compare image-wise prediction module 202 of FIG. 2). Each image of the input image sequence is processed according to framework 300 of FIG. 3 to generate an image-wise BCC prediction score. In the exemplary embodiment, each received 1000×1000 pixels RCM image (see reference numeral 302 of FIG. 3 for an example) is resized to 224×224 pixels (see reference numeral 304 of FIG. 3). The resizing operation may be performed by the processing module. In the exemplary embodiment, the processing module is configured to feed (or transmit) the plurality of resized 224×224 pixels RCM images to a 1-cut model 306 (the 1-cut model is an example of a third classification model described in various exemplary embodiments). The 1-cut model 306 has been trained to provide a 1-cut full image prediction result (the 1-cut full image prediction result is an example of a preliminary whole image prediction result described in various exemplary embodiments). See reference numeral 307 of FIG. 3.

In the exemplary embodiment, the processing module is further configured to process/divide each 1000×1000 pixels RCM image 302 (corresponding to a whole image described in various exemplary embodiments) equally into four 500×500 pixels images (these images from the whole image are examples of a plurality of first sub-sections described in various exemplary embodiments). For example, each input RCM image may be divided into four images or first sub-sections, these images being the up-left (UL), up-right (UR), bottom-left (BL), and bottom-right (BR) portions of the 1000×1000 pixels RCM whole image. In the exemplary embodiment, each 500×500 pixels image is resized to 224×224 pixels. See reference numeral 308 of FIG. 3. The resizing operation may be performed by the processing module.

In the exemplary embodiment, the processing module is configured to feed (or transmit) the four resized 500×500 pixels images to a respective 4-cut model 310 (the 4-cut model is an example of a first classification model described in various exemplary embodiments). The 4-cut model 310 has been trained to process the four resized 500×500 pixels images and to provide four 4-cut prediction results, with one 4-cut prediction result generated for each of the four 500×500 pixels images. In FIG. 3, the prediction result of an UL sub-section is output at numeral 311.

In the exemplary embodiment, the processing module is further configured to further process/divide each of the 4-cut 500×500 pixels image 308 into four 250×250 pixels images (e.g., a BR first sub-section 313 of the whole image 302 is divided further into UL, UR, BL, BR portions), i.e., to produce sixteen 250×250 pixels images (these images from the first sub-sections are examples of a corresponding plurality of second sub-sections described in various exemplary embodiments). For example, an UL first sub-section of the whole image would have a corresponding plurality of four second sub-sections (i.e., its UL, UR, BL, BR second sub-sections). In the exemplary embodiment, each 250×250 pixels image is resized to 224×224 pixels. See reference numeral 312 of FIG. 3. The resizing operation may be performed by the processing module.

In the exemplary embodiment, the processing module is configured to feed (or transmit) the sixteen resized 250×250 pixels images to a respective 16-cut model 314 (the 16-cut model is an example of a second classification model described in various exemplary embodiments). The 16-cut model 314 has been trained to process the sixteen resized 250×250 pixels images and to provide sixteen 16-cut prediction results, with one 16-cut prediction result generated for each of the sixteen resized 250×250 pixels images. In FIG. 3, the prediction result of a 16-cut model for one second sub-section is output at numeral 315.

In the exemplary embodiment, to generate an image-wise BCC prediction score, the processing module is further configured to ensemble the 21 prediction results obtained (one 1-cut image prediction result, four 4-cut image prediction results and sixteen 16-cut image prediction results) using a hierarchical ensemble structure of the plurality of classification models, namely, the 4-cut model (or the first classification model), the 16-cut model (or the second classification model), and the 1-cut model (or the third classification model).

In FIG. 3, an exemplary illustration is provided for one block or one block structure of the ensemble structure. The 4-cut prediction result 311 for the UL portion of the 1000×1000 pixels RCM image and its corresponding four 16-cut prediction results e.g., 315 are fed (or transmitted) to a support vector machine (SVM) model 316 (the SVM model 316 is an example of a block SVM described in various exemplary embodiments). The SVM model 316 is denoted as 4-16 SVM. In the exemplary embodiment, the 4-cut model 310, the 16-cut model e.g., 314 and the 4-16 SVM 316 form a UL 416 block 318. In the exemplary embodiment, the inventors recognise that the contribution of each second sub-section (e.g. each of the sixteen images or 16-cut images) to the image-wise prediction score or result may be substantially the same. The same assumption may also apply to each first sub-section (e.g. each of the four images or 4-cut images). The inventors further recognise that that typically, the size of the BCC lesions may be substantially larger than the field of view of a RCM and therefore, after an image augmentation process (e.g., in training the classification models), the probability of BCC lesions appearing in each sub-section may be about the same. The inventors thus recognise that, with such understanding, it is sufficient to train and provide one 4-cut model (trained to process a first sub-section) and one 16-cut model (trained to process the corresponding second sub-sections) in each block e.g., UL 416 block 318, and for use in all blocks 318, 320, 322, 324. In the exemplary embodiment, the output generated by the 4-16 SVM 316 is output at numeral 319 (the output of the block 318 is an example of a respective block prediction result corresponding to each first sub-section described in various exemplary embodiments. In this case, the respective block prediction result is with regard to, or corresponding to, the UL first sub-section or UL 4-cut image from the whole image 302).

In the exemplary embodiment, a SVM is provided as a machine learning linear model for classification and can function to map data to a high-dimensional feature space so that data points can be categorized/classified. For example, a separator between the categories/classes can be found, and the data can be transformed in such a way that the separator could be drawn as a hyperplane.

In the exemplary embodiment, it is recognised that the output range of each block SVM e.g., 316 is (−∞, +∞). In the exemplary embodiment, a sigmoid function

$$S ( x ) = \frac{1}{1 + {e}^{- x}}$$

is used to normalise each block SVM output so that each output is under the same scale [0, 1] as the 1-cut model output. Thus, in the exemplary embodiment, the sigmoid function may map the block SVM output values into values between 0 and 1, and therefore mapping predictions to probabilities.

In the exemplary embodiment, in a substantively similar manner to the above for the UL 416 block 318, the respective block prediction results corresponding to the other first sub-sections, i.e., the results 321, 323, 325 of UR, BL, and BR 416 blocks 320, 322, 324 respectively of FIG. 3 are generated.

In the exemplary embodiment, the four 416 block outputs 319, 321, 323, 325 and the 1-cut model prediction result 307 (i.e., the output of the 1-cut model 306) are fed to another separate SVM 326 (the SVM 326 is an example of an image-wise stage SVM described in various exemplary embodiments). The SVM 326 is denoted as 1-4 SVM. That is, the preliminary whole image prediction result (i.e., the output 307 of the 1-cut model 306) and a plurality of the respective block prediction results each corresponding to the first sub-sections of the whole image are fed to the 1-4 SVM 326.

The sigmoid function

$$S ( x ) = \frac{1}{1 + {e}^{- x}}$$

is used to normalise the SVM output of the 1-4 SVM 326 to obtain/generate an image-wise BCC prediction score 328 (or the probability of BCC) for the input image. In the exemplary embodiment, the image-wise BCC prediction score 328 is in the form of a value between 0 (zero) to 1 (one), both inclusive.

In the exemplary embodiment, in use, with the framework 300, an image-wise BCC prediction score 328 may be provided by the image-wise prediction module of the prediction module for each RCM image of an input image sequence. With the hierarchical ensemble structure, information loss from resizing of a whole image (i.e., with the use of only a 1-cut model) may be usefully minimised.

Therefore, in this exemplary embodiment, a hierarchical ensemble structure is provided that comprises at least one block structure, each block structure corresponding to a respective first sub-section processed from a whole image; in the each block structure, a first classification model is provided to process the respective first sub-section and a second classification model is provided to process a corresponding plurality of second sub-sections, the corresponding second sub-sections being processed from or corresponding to the first sub-section of the block structure; the hierarchical ensemble structure further comprising a first classification model; wherein the each block structure is arranged to provide a respective block prediction result and the first classification model is arranged to provide a preliminary whole image prediction result. The hierarchical ensemble structure may further comprise, in each block structure, a block SVM; wherein an output of the first classification model and an output of the second classification model are transmitted to the block SVM to generate the respective block prediction result. The hierarchical ensemble structure may further comprise an image-wise stage SVM; wherein the preliminary whole image prediction result of the first classification model and a plurality of respective block prediction results each corresponding to the first sub-sections processed from the whole image are transmitted to the image-wise stage SVM to generate an image-wise BCC prediction score or a probability of BCC of the whole image.

In the exemplary embodiment, the image-wise BCC prediction score 328 generated is used to determine a sequence-wise BCC prediction score (corresponding to a BCC classification of the input image sequence described in various exemplary embodiments).

In a typical RCM scanning, a sequence of RCM images at consecutive depths (for example, each image obtained at an increasing depth with respect to a subject skin surface) may be provided. In the exemplary embodiment, to compute a sequence-wise BCC prediction score for an entire RCM input image sequence, the image-wise BCC prediction scores along depth (i.e., the image-wise BCC prediction scores obtained for the plurality of RCM images of the input image sequence) are treated as 1-D input data. An example of such 1-D input data in one exemplary embodiment is illustrated in a graphical representation in FIG. 4A.

FIG. 4A is a graph showing exemplary image-wise BCC prediction scores for a plurality of images from a RCM (reflectance confocal microscopy) input image sequence along a depth of the RCM input image sequence. The graph shown in FIG. 4A is an exemplary probability curve of a RCM input image sequence obtained from a BCC patient. For example, it can be observed at FIG. 4A that the probabilities of BCC of the images between the relative depth of 10 to 30 μm are between 0.8 to 1.

In the exemplary embodiment, a sequence-wise BCC prediction score evaluation method based on image-wise BCC prediction scores along depth (as shown below) is used:

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$

In the above equation, Sₐᵢ stands for the area of each consecutive part between BCC score values 0.8 to 1, and similarly, S_{bj} and S_{ck} stand for areas of each consecutive part between score values 0.6 to 0.8 and score values 0.4 to 0.6 respectively. lₐᵢ, l_{bj} and l_{ck} stand for the corresponding length of Sₐᵢ, S_{bj}, and S_{ck}.

In the exemplary embodiment, a positive small value constant θ=0.01 may be added to the equation to prevent a case of having B=ln 0, i.e.,

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} + θ ) .$$

With the above relationship, it can be observed that more weightage is given to portions having higher BCC score values as compared to portions having lower BCC score values. Similar to image-wise BCC probabilities, the higher the B score of the sequence-wise prediction is, the more confidence there can be that BCC lesions exist within an input image sequence. For the image-wise BCC prediction output, when the probability of BCC is high, e.g., the probability is 0.9, one may be “substantially confident” that there are BCC lesions within an input image sequence. When the probability is 0.7, one may be “not so sure” that there are BCC lesions within an input image sequence. The confidence is lowered further when the probability is 0.5. In the exemplary embodiment, the level of confidence is not linear with the value of probability. Therefore, in the exemplary embodiment, different weights are assigned to probabilities in different portions/intervals to achieve better classification performance.

An illustration of data for sequence-wise BCC score calculation in one exemplary embodiment is shown in FIG. 4B. FIG. 4B is a graph illustrating data for calculating a sequence-wise BCC prediction score in an exemplary embodiment.

In the exemplary embodiment, the sequence-wise BCC score is calculated by the following equation:

$$B = ln ( {S}_{a 1} {e}^{2 {l}_{α 1}} + {S}_{b 1} {e}^{{l}_{b 1}} + {S}_{c 1} {l}_{c 1} + {S}_{c 2} {l}_{c 2} + {S}_{c 3} {l}_{c 3} + θ ) .$$

Referring to FIG. 1, in an exemplary embodiment, the processing module 104 of the system 100 may further be configured to perform training of the plurality of classification models using at least two training groups of images. Such training groups of images may be captured and compiled by the confocal microscopy device 102.

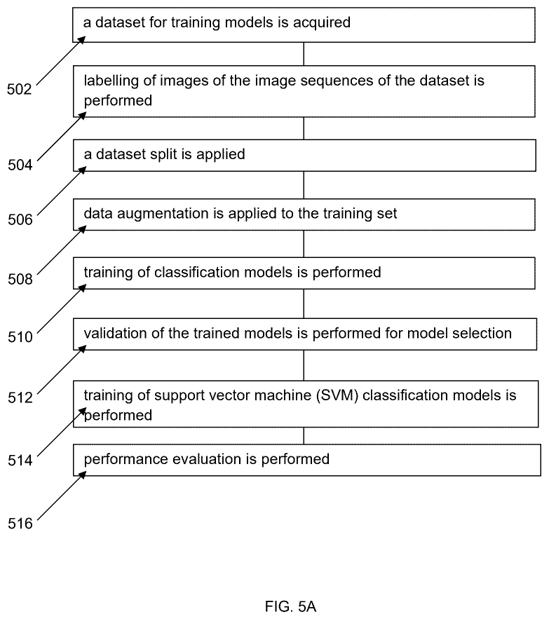

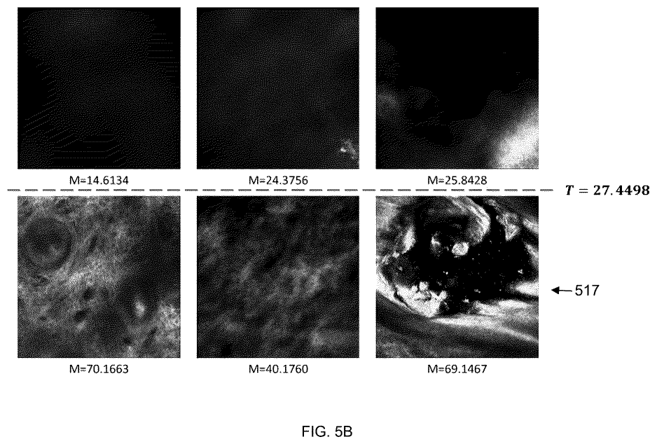

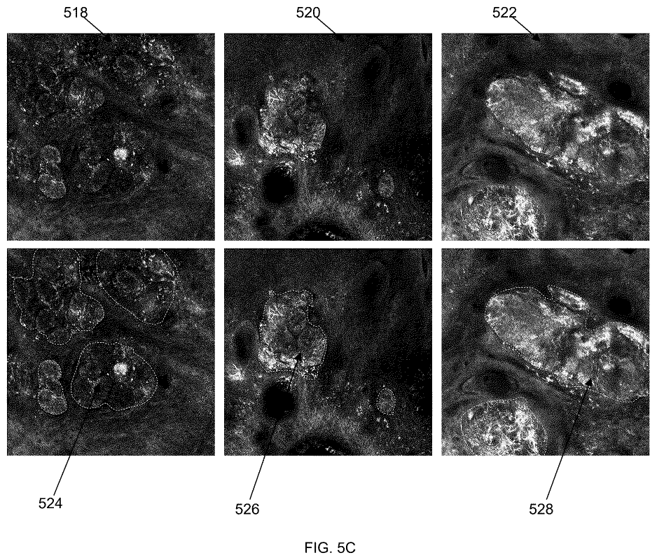

FIG. 5A is a schematic flowchart illustrating a method of training models in an exemplary embodiment. The models are deep learning models and are classification models. In the exemplary embodiment, one or more steps of the method are computer-implemented.

Alternatively, the method is a computer-implemented method. In the exemplary embodiment, the method may be implemented by a processing module (e.g., see processing module 104 described with reference to FIG. 1). For ease of reference, like naming conventions are used for exemplary implementations of similar modules as described in FIG. 1.

In the exemplary embodiment, the processing module is configured to perform training of a plurality of classification models (e.g., a 4-cut model, 16-cut model, and a 1-cut model, corresponding to a first classification model, a second classification model, and a third classification model respectively as described in various exemplary embodiments) and SVM models (e.g., a 4-16 SVM model and a 1-4 SVM model, corresponding to a block SVM and an image-wise stage SVM respectively as described in various exemplary embodiments).

In the exemplary embodiment, at step 502, a dataset for training models (or training dataset) is acquired. In the exemplary embodiment, the dataset for training comprises a plurality of image sequences, each comprising a plurality of images obtained at pre-determined/consecutive depths, obtained/captured by a confocal microscopy device (e.g., compare confocal microscopy device 102 described with reference to FIG. 1).

In the exemplary embodiment, the confocal microscopy device may be a commercialised confocal microscopy system/device, VivaScope™ 3000, which is used for confocal image scanning. In the exemplary embodiment, when in use, the confocal microscopy device has a spatial resolution at cellular level of 1.25 μm. For vertical image scanning, the confocal microscopy device has 5.0 μm vertical resolution, and the scanning speed is larger than 6 frames per second. A 30-frame sequence scanning can therefore be finished within 5 seconds. For a single scanning frame, the field of view (FOV) is 750×750 μm², which the inventors recognise is sufficient to capture important BCC features for classification. The penetration depth is 150 μm, which the inventors recognise is sufficient to reach the dermis layer of facial skin, which is typically one of the prevalent sites of BCC. This depth is also close to stratum basale, where BCC typically originates. Therefore, in the exemplary embodiment, the confocal microscopy device is recognised by the inventors to be useful in the early detection of BCC.

In the exemplary embodiment, for dataset acquisition for training of classification models, image sequences for subjects with BCC lesions and for subjects without any diagnosed skin cancers are acquired, i.e., two training groups of images are obtained and used for training of classification models. In the exemplary embodiment, subjects with BCC lesions and subjects without any diagnosed skin cancers are grouped into a BCC group and an NS (normal skin) group respectively. The NS group may also be denoted as a non-BCC group. Thus, there are at least two training groups of images that can be used.

In the exemplary embodiment, the at least two training groups of images therefore comprise a BCC training group of images and a non-BCC training group of images. In the exemplary embodiment, the at least two training groups of images may be stored in a deep learning database. In the exemplary embodiment, the BCC training group of images comprises images of known BCC subjects arranged as one or more BCC image sequences and the non-BCC training group of images comprises images of normal subjects (or non-BCC subjects or normal healthcare patients or normal skin subjects) arranged as one or more non-BCC image sequences.

In one exemplary embodiment, more than 485 image sequences (e.g., RCM scanning sequences or RCM image sequences) for 182 subjects with diagnosed BCC lesions and 96 subjects without any diagnosed skin cancers are acquired. The subjects were volunteers recruited from the Singapore National Skin Centre. These subjects are grouped into the BCC group and the NS group respectively. In the exemplary embodiment, the image sequences described above form a so-called Singapore National Skin Centre (NSC) dataset. From the 182 subjects, 258 image sequences with 8864 images in the BCC group, and 232 image sequences with 7362 images in the NS group are collected.

In the exemplary embodiment, at step 504, labelling/annotation of images of the image sequences of the dataset is performed. In the exemplary embodiment, the labelling may be performed by a healthcare professional, for example, a professional dermatologist through manual examination of the images. During the labelling, each image of the image sequences obtained at step 502 is examined and is given a label, indicating whether each image shows observable BCC features. Such BCC features may include, for example, BCC lesions. If an image shows observable BCC features, the image may be given one label. If an image does not show observable BCC features, the input image may be given another label, distinct from the label for images with observable BCC features.

Through manual examination by professional dermatologists, 5418 images in the BCC group with observable BCC features are labelled as “B”. All images in the NS group are labelled as “N”. These 5418 B images and 7362 N images form a dataset (comprising two training groups of images) that is used for training of classification models. For example, the dataset is used for the training of 1-cut, 4-cut, and 16-cut models for image-wise prediction (compare framework 300 of FIG. 3).

FIG. 5C shows examples of BCC features that can be used for classification of BCC under confocal microscopy scanning. Three images 518, 520 and 522 are shown. Such images may be examples from the BCC training group of images. Significant/important BCC features (e.g., major BCC lesions 524, 526, 528 (marked out with white dotted lines)) can be identified from these images. For example, tumour islands, peripheral palisading and clefting can be identified.

Returning to FIG. 5A, in the exemplary embodiment, at step 506, a dataset split is applied. The dataset is split into a training set, a validation set, and a test set at a ratio of 8:1:1 under the unit of patient such that images in these subsets (the training set, the validation set, or the test set) are independent. The split may be seen as a k-fold cross-validation split applied to the dataset (where k is a value) through different combinations of the subsets. In the exemplary embodiment, the split may be seen as a 10-fold cross-validation split (i.e., where the value of k is 10 is applied). The inventors recognise that to make use of the depth information provided by confocal microscopy, an input unit to obtain a final prediction/classification from the system (compare system 100) is one input image sequence, e.g., one confocal scanning sequence. Therefore, the data split of the exemplary embodiment is based on image sequences (or unit of patient) instead of each individual image.

As such, in the exemplary embodiment, the processing module (compare processing module 104 of FIG. 1) may be configured to split the training groups of images into a training set, a validation set, and a test set at a ratio of 8:1:1.

In the exemplary embodiment, at step 508, data augmentation is applied to the training set. Images in the training set are augmented to 20,000 images in each category, BCC and non-BCC (40,000 in total) to make a more balanced training set for the deep learning models.

In the exemplary embodiment, in the process of applying data augmentation, filtering of images in the training set is applied to determine which images are suitable for the augmentation. For investigation of the appropriate filter, a plurality of images (over 20,000 RCM images of skin) are collected or obtained/acquired by a confocal microscopy device. An average signal intensity (M) and a standard error (SE) thereof are measured. A threshold signal intensity (T) is then calculated by the formula T=M−SE. Based on the threshold signal intensity calculated, images in the training set are filtered such that images with an average grayscale level larger than the value of T undergo data augmentation. The filtering of images in the training set usefully ensures that the images in the training set (which are used for training classification models) provide a sufficient level of information (e.g., structural information) for model training purposes.

As such, in the exemplary embodiment, the processing module (compare processing module 104 of FIG. 1) is configured to perform data augmentation of the BCC training group of images and the non-BCC training group of images based on a threshold value of an image average signal intensity. The processing module is configured to apply data augmentation to the BCC training group of images and the non-BCC training group of images with measured average grayscale level being larger than the threshold image average signal intensity. On the other hand, the processing module is configured to not apply data augmentation to those BCC training group of images and the non-BCC training group of images with measured average grayscale level being equal to or smaller than the threshold image average signal intensity.

In the exemplary embodiment, with the more than 20,000 RCM images of skin, the average signal intensity is measured to be M=43.0578 (under 8-bit grayscale range 0 to 255), with its standard error being SE=15.6080. A threshold T=M−SE=27.4498 is set and images with an average grayscale level larger than the value of T are used for augmentation.

FIG. 5B is an exemplary set of images below and above a pre-determined threshold signal (T). FIG. 5B shows images below the threshold (top row) and images above the threshold (bottom row). The threshold line T=27.4498 is shown separating the two rows of images. As such, the bottom row 517 can be used for augmentation.

Returning to FIG. 5A, at step 508, data augmentation is applied to the filtered training set obtained. In the exemplary embodiment, applying data augmentation to the filtered training set may usefully increase the number of images available for training of models by creating and including modified versions of the input images in the training set. Data augmentation may also usefully balance the number of images under different categories in a training set. In the exemplary embodiment, augmentation methods may include, for example, random brightness change (e.g., of 90% to 110%), random contrast change (e.g., of 90% to 110%), random vertical/horizontal flip, random degree rotation and random cropping. If 0-padding is introduced in a random degree rotation procedure, the image may be cropped in a following random cropping procedure. In the exemplary embodiment, stretching is not applied, so that the morphology of the scanned tissue shown in the images is preserved. In the exemplary embodiment, blurring is also not included, as partially blurred confocal images are typically rare, and will typically not be considered as valid in clinical cases for diagnosis.

In the exemplary embodiment, for training the plurality of classification models of the hierarchical ensemble structure, the augmented images of BCC subjects are grouped in the same image sequence as their respective original BCC images, in the training set. The augmented images of BCC may share a significant resemblance or similarity with the original BCC images, and with other augmented images of BCC subjects that are based on the same original BCC images (i.e., same origins). In the exemplary embodiment, it is desired that a robust classification model delivers favourable performances with the training group(s) of images, as well as with new images (or unseen data). This is referred to as generalisation of a model. Therefore, in use, including and grouping the augmented images of BCC subjects in the same image sequence as their respective original BCC images for a training set can desirably improve the training and evaluation of the models. For the validation set and testing set, no augmented BCC images are included, e.g., to have real data for validation and testing rather than the data being supplemented with augmented data.

In the exemplary embodiment, after data augmentation is applied to the training set (see step 508), the data in the training set (whole images) is used for 1-cut model training (corresponding to training a third classification model described in various exemplary embodiments) and is denoted as a 1-cut dataset (or a whole image dataset). Based on the 1-cut dataset (whole images of the whole image dataset), each image is equally divided to four 500×500 pixels images (corresponding to a plurality of first training sub-sections) to form a 4-cut dataset (or a first training sub-sections dataset). The dividing/processing can be performed by the processing module. Similarly, each image in the 4-cut dataset is further divided to four 250×250 pixels images (corresponding to a corresponding plurality of second training sub-sections) to form a 16-cut dataset (or a second training sub-sections dataset). The dividing/processing can be performed by the processing module. The 4-cut dataset and the 16-cut dataset are used to train the 4-cut model and 16-cut model respectively (corresponding to training a first classification model to process the first sub-sections as described in various exemplary embodiments and training a second classification model to process the second sub-sections as described in various exemplary embodiments). In the exemplary embodiment, all images in the 4-cut and 16-cut datasets follow the same label as their corresponding whole image in the 1-cut dataset.

In the exemplary embodiment, at step 510, training of classification models is performed. A residual neural network, for example, a matured image classification model of ResNet 101 is adopted for training a plurality of classification models (e.g., the first classification model, the second classification model, and the third classification model) of a hierarchical ensemble structure (compare the prediction module 106 of FIG. 1 and the framework 300 of FIG. 3). In the exemplary embodiment, ResNet 101 model is used for 1-cut, 4-cut, and 16-cut model training. ResNet 101 is a convolutional neural network (CNN) that is 101 layers deep, and can be trained and used to classify images into a plurality of object categories. Thus, at least one classification model of the exemplary embodiment may be trained to each process the whole image, the first sub-sections and the corresponding second sub-sections (corresponding to the first sub-sections) of each image of an input image sequence (e.g., captured and compiled by the confocal microscopy device 102 of FIG. 1). In the exemplary embodiment, in training each classification model based on the at least two training groups of images, the problem to be solved may be set as a binary classification problem, and thus, the number of class is set as 2. With such a setting, the output of a trained classification model is a probability. For example, in a 2-class problem, there may be class A and class B, and the model's output is the probability of an input image belonging to class A and class B, P(A) and P(B), where P(A)+P(B)=1. The final classification result on whether the input image belongs to A or B depends on a probability threshold. With different threshold settings, the same output P(A), P(B) could result in different classification results. Further, in the exemplary embodiment, an exponential decay learning rate is used to replace the default learning rate, with the initial learning rate set as 0.001, decay steps set as 10000, and an exemplary decay rate of 0.9 is used. An Adam optimizer with the above-mentioned exponential decay learning rate is applied. A categorical cross-entropy is used as the loss function, and together with average accuracy on both the training set and the validation set, to monitor the training progress. These settings or tunings (or hyperparameters embedded in codes) may be used for the ResNet 101 model training using available programming toolkits such as TensorFlow (a deep learning platform that provides tools to build a deep learning model and is a Python toolkit that is publicly available online).

In the exemplary embodiment, training is performed on each of the plurality of classification models (e.g., the first classification model, the second classification model, and the third classification model) of the hierarchical ensemble structure. In the exemplary embodiment, the training of each 1-cut, 4-cut, and 16-cut model is repeated a plurality of times, e.g., 20 times with 4000 epochs. Different combination of hyperparameters may be used at each epoch and the final model selection of a 1-cut model, 4-cut model and a 16-cut model for the hierarchical ensemble structure is based on selecting a 1-cut model, a 4-cut model and a 16-cut model with the best validation set performance (see step 512) and trained with a specific combination of hyperparameters.

In the exemplary embodiment, at step 512, validation of the trained models is performed. In the exemplary embodiment, at step 512, the validation set (obtained at step 506) is used to evaluate trained models, e.g., by obtaining and evaluating image-wise BCC prediction scores (corresponding to a probability of BCC or an image-wise BCC prediction score described in various exemplary embodiments) calculated by the trained models for each image (of an input image sequence) in the validation set. In the exemplary embodiment, the model with the best validation set performance (e.g., the model which generates the most number of image-wise BCC prediction scores which are in agreement with the image labels) and trained with a specific combination of hyperparameters (see step 510) is selected to be used as the 1-cut model, 4-cut model, and the 16-cut model.

The plurality of trained classification models can then be arranged and used in the hierarchical ensemble structure for image-wise prediction (compare framework 300 of FIG. 3), i.e., predicting or determining a BCC probability of each image in an image sequence.

In the exemplary embodiment, at step 514, training of SVM models (i.e., a block SVM and an image-wise stage SVM, or in exemplary embodiments, a 4-16 SVM model and a 1-4 SVM model) is performed. In the exemplary embodiment, the SVM model training is based on the 1-cut, 4-cut, and 16-cut model image-wise BCC prediction outputs/scores obtained using the training set and validation set. In the exemplary embodiment, the kernel functions of both a 4-16 SVM model (corresponding to a block SVM described in various exemplary embodiments) and a 1-4 SVM model (corresponding to an image-wise stage SVM model described in various exemplary embodiments) are set as a linear function, for classification purposes.

FIG. 5D is an exemplary illustration showing training of SVM models in an exemplary embodiment.

In the exemplary embodiment, data from the whole image dataset (e.g. 1-cut data from the 1-cut dataset) is processed by the third classification model (e.g. the 1-cut model) and data from the first training sub-sections dataset (e.g. 4-cut data from the 4-cut dataset) is processed by the first classification model (e.g. the 4-cut model). The prediction results of the third classification model and the first classification model are used to train an image-wise stage SVM.

In the exemplary embodiment, as shown in FIG. 5D, a 1-cut image or a whole image 530 and its corresponding first sub-sections or 4-cut images 532, 534, 536, 538 (i.e. the UL, BL, UR, BR respectively of the whole image 530) are processed by a 1-cut model and a 4-cut model respectively to provide prediction results that are used to train an image-wise stage SVM or a 1-4 SVM model 540.

In the exemplary embodiment, the 1-4 SVM model 540 (compare 1-4 SVM 326 described with reference to FIG. 3) is trained by receiving, as an input, a 1-cut model preliminary whole image prediction result 0.999778 (see numeral 542) obtained based on the whole image 530 from the 1-cut dataset of the training set. The 1-4 SVM model 540 also receives, as inputs, 4-cut model prediction results 0.781062 (see numeral 544), 0.998687 (see numeral 546), 0.993205 (see numeral 548), and 0.994189 (see numeral 550), each obtained based on corresponding data from the 4-cut dataset of the training set. The 1-cut model is used to process further remaining whole image data from the 1-cut dataset from the training set and the 4-cut model is used to process the corresponding 4-cut data from the 4-cut dataset from the training set to provide prediction results that are iteratively used to train the 1-4 SVM model 540. The 1-4 SVM model 540 can be validated in a similar manner using the validation set.

In the exemplary embodiment, data from the first training sub-sections dataset (e.g. 4-cut data from the 4-cut dataset) is processed by the first classification model (e.g. the 4-cut model) and data from the second training sub-sections dataset (e.g. 16-cut dataset) is processed by the second classification model (e.g. the 16-cut model). The prediction results of the first classification model and the second classification model are used to train a block SVM.

In the exemplary embodiment, as shown in FIG. 5D, a 4-cut image 552 and its corresponding second sub-sections or 16-cut images 554, 556, 558, 559 (i.e. the UL, BL, UR, BR respectively of the 4-cut image 552) are processed by a 4-cut model and a 16-cut model respectively to provide prediction results that are used to train an block SVM or a 4-16 SVM model 560.

In the exemplary embodiment, the 4-16 SVM model 560 (compare 4-16 SVM 316 described with reference to FIG. 3) is trained by receiving, as an input, a 4-cut model prediction result 0.993205 (see numeral 562) obtained based on the 4-cut image 552 from the 4-cut dataset of the training set. Compare the 4-cut image 536 and the 4-cut model prediction result at numeral 548. The 4-16 SVM model 560 also receives, as inputs, 16-cut model prediction results 0.994391 (see numeral 564), 0.999966 (see numeral 566), 0.995002 (see numeral 568), and 0.999986 (see numeral 570), each obtained based on corresponding data from the 16-cut dataset of the training set. The 4-cut model is used to process further remaining 4-cut image data from the 4-cut dataset from the training set and the 16-cut model is used to process the corresponding 16-cut data from the 16-cut dataset from the training set to provide prediction results that are iteratively used to train the 4-16 SVM model 560. The 4-16 SVM model 560 can be validated in a similar manner using the validation set.

Therefore, in the exemplary embodiment, the 1-4 SVM model 540 and the 4-16 SVM model 560 are each trained with data via five inputs.

Returning to FIG. 5A, in the exemplary embodiment, at step 516, performance evaluation is performed. For sequence-wise performance evaluation, the original RCM scanning sequences (image sequences) are used for testing (without image exclusion, such as filtering based on a measured average grayscale level of images, and regardless of image qualities or image labels). From the training groups of images, scanning sequences from BCC patients are labelled as BCC sequences if there is a scanning sequence that contains at least 1 B-labelled image. Scanning sequences from BCC patients with no observable BCC features throughout a whole image sequence (i.e., contain no B-labelled image) were excluded from the sequence-wise performance evaluation, as such sequences cannot be determined by healthcare professionals (e.g., dermatologists) on whether any BCC lesions are included in the respective scanning area when obtaining those image sequences. All scanning sequences from control subjects are labelled as NS sequences. The sequence-wise performance evaluation may be performed, e.g., by obtaining and evaluating sequence-wise BCC prediction scores (corresponding to a BCC classification of an image sequence described in various exemplary embodiments) generated via the trained models.

In the exemplary embodiment, it will be appreciated that for further enhancing the robustness of the models, and for further validation, more datasets (e.g., more image sequences/more RCM scanning sequences) may be collected and used for training the models.

In the exemplary embodiment, after the classification models (e.g. the first classification model, the second classification model, the third classification model, the block SVM and the image-wise stage SVM) are trained, the performances of these classification models can used to evaluate the capability of the classification models.

In an exemplary embodiment, model training and testing are performed using a 10-fold cross-validation scheme to provide a comprehensive performance evaluation. In the exemplary embodiment, the system and method for classification of BCC is tested on two RCM datasets, namely, one dataset from subjects recruited by the Singapore National Skin Centre, i.e., the so-called NSC dataset (see step 502 described with reference to FIG. 5A), and a public MSKCC (Memorial Sloan Kettering Cancer Centre) RCM dataset.

For the MSKCC dataset, an image split for 10-fold cross-validation (see step 506 described with reference to FIG. 5A) is based on a sequence unit (or image sequence) instead of a patient unit, as patient information is not provided. From the MSKCC dataset, images that are labelled with S (suspicious), N (normal skin), and NB (not BCC) within BCC sequences are excluded from the training set for training classification models (see step 510 described with reference to FIG. 5A) and testing set. Images labelled with B (BCC) and S (suspicious) within a non-BCC sequence are also excluded from the training set and testing set. When performing sequence-wise testing (see step 516 described with reference to FIG. 5A), BCC sequences with no B-label images and non-BCC sequences with B-label images are excluded from the testing set. All remaining images within qualified RCM image sequences are preserved regardless of their labels and image qualities. Outputs of trained classification models for image-wise prediction based on RCM image sequences are resized to a 1×36 vector before being transmitted to trained classification (SVM) models to obtain a sequence-wise BCC prediction score. Other image pre-processing methods (e.g., see step 508 described with reference to FIG. 5A) and parameter settings are substantially the same as when the classification models are trained based on the NSC dataset (i.e., based on the method described with reference to FIG. 5A).

In the exemplary embodiment, with the above, from the MSKCC dataset, 95 BCC sequences with 2705 images and 131 NS sequences with 6187 images are available for image-wise training and testing of the classification models. There is a same number of BCC and NS sequences with no image exclusions used for sequence-wise testing.

In the exemplary embodiment, the performances at an image-wise BCC prediction stage (wherein a probability of BCC is generated for each image of an input image sequence e.g., by an image-wise prediction module 202 described with reference to FIG. 2) under the 10-fold cross-validation scheme are displayed in Table 1. Table 1 also shows enhancements provided by using a hierarchical SVM ensemble method (using a hierarchical ensemble structure with SVM models of exemplary embodiments) as compared to using only a 1-cut model. The values are shown in terms of AUC. AUC refers to the Area Under Curve for a Receiver Operator Characteristic (ROC) curve, while the ROC is a probability curve that is an evaluation metric for binary classification problems. For the NSC dataset, the hierarchical SVM ensemble provides an average of 1.6139% AUC enhancement over the mean 1-cut model performance of 97.2955%. For the MSKCC dataset, the hierarchical SVM ensemble provides an average of 3.8018% AUC enhancement over the 1-cut model performance of 84.6293%.

TABLE 1

| | NSC | | MSKCC | |
|---|---|---|---|---|
| Group No. | 1-cut model | SVM | 1-cut model | SVM |
| 1 | 98.1511 | 99.9984 | 82.2814 | 83.8005 |
| 2 | 93.3006 | 99.2960 | 85.5371 | 86.2943 |
| 3 | 98.2302 | 99.2764 | 77.2353 | 83.3589 |
| 4 | 96.0970 | 98.6068 | 85.8183 | 89.2610 |
| 5 | 97.3301 | 98.2253 | 80.3342 | 85.0365 |
| 6 | 97.9375 | 97.7406 | 84.5286 | 89.6130 |
| 7 | 98.9030 | 99.2298 | 88.2508 | 93.0497 |
| 8 | 96.8438 | 98.9661 | 87.1777 | 92.9390 |
| 9 | 97.8005 | 98.4833 | 88.0677 | 89.8029 |
| 10 | 98.3612 | 99.2715 | 87.0615 | 91.1547 |
| Mean | 97.2955 | 98.9094 | 84.6293 | 88.4311 |

Next, performances at a sequence-wise BCC prediction stage (wherein a BCC classification is generated for an image sequence e.g., by the sequence-wise prediction module 204 described with reference to FIG. 2, based on the probability of BCC generated for each image of the image sequence e.g., by the image-wise prediction module 202) in one exemplary embodiment are evaluated.

In the exemplary embodiment, in the NSC dataset, the RCM image sequences are scanned via 2 scanning methods, namely, obtaining 32 image sequences with depth scanning step size of 3.26 μm and obtaining 36 image sequences with depth scanning step size of 4.56 μm. In the exemplary embodiment, the image-wise probabilities of BCC obtained for the image sequences scanned by the former method (32 image sequences) are resized to 36 by interpolation so that the method of obtaining BCC classification at the sequence-wise BCC prediction stage (e.g., implemented by the sequence-wise prediction module 204 described with reference to FIG. 2, compare FIGS. 4A and 4B) can be directly applied.

In the exemplary embodiment, in the MSKCC dataset, the RCM image sequences range from 23 to 83 images. In the exemplary embodiment, image-wise probabilities of BCC for the MSKCC RCM image sequences are normalised to 36 using downsizing or interpolation before obtaining BCC classifications at the sequence-wise BCC prediction stage.

The performances at a sequence-wise prediction stage in the exemplary embodiment under the 10-fold cross-validation scheme is displayed in Table 2. In the NSC dataset, with the system of the exemplary embodiment, a close-to-perfect performance, with an average AUC of 99.9227%, was achieved. It is observed that in 9 groups of the 10-fold cross-validation, with the system of the exemplary embodiment, a 100% AUC was achieved. As for the public MSKCC dataset, an average AUC of 92.4337% was achieved. The inventors recognise that this may be about 2.3337% better than using a single CNN ResNet 34 model reported elsewhere.

TABLE 2

| Group No. | NSC | MSKCC |
|---|---|---|
| 1 | 100 | 94.4248 |
| 2 | 100 | 87.7911 |
| 3 | 100 | 87.2571 |
| 4 | 100 | 97.6288 |
| 5 | 100 | 92.4125 |
| 6 | 99.2274 | 89.9285 |
| 7 | 100 | 94.5593 |
| 8 | 100 | 92.3520 |
| 9 | 100 | 89.2225 |
| 10 | 100 | 98.7654 |
| Mean | 99.9227 | 92.4337 |

In the exemplary embodiment, as shown in Table 1 and Table 2, the performance of the system for BCC classification for different datasets at the image-wise prediction stage and the sequence-wise prediction stage can be relatively high, e.g., above 88% and 90%.

In an exemplary embodiment, a determined BCC classification of an input image sequence may be transmitted to an output device (compare output device 108 of FIG. 1). The output device can provide/present the determined BCC classification of the input image sequence to a user. In the exemplary embodiment, the output device may also provide visualisation of BCC classification using images.

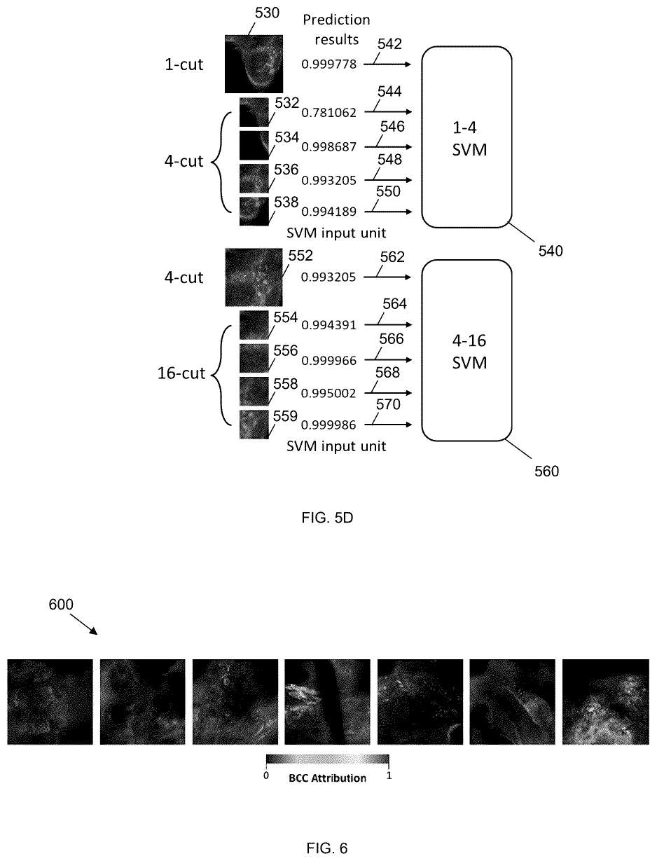

FIG. 6 is a set 600 of heatmaps generated by a self-embedded attribution map in an exemplary embodiment. In the exemplary embodiment, the self-embedded attribution map and heatmaps are graphical/visual representations of the determined probability of BCC of each image of an image sequence described in various exemplary embodiments.

In the exemplary embodiment, the inventors recognise that one further benefit of using a hierarchical ensemble structure, e.g., in a system for classification of BCC 100 described with reference to FIG. 1 and the framework 300 of FIG. 3, is that the system for classification of BCC can directly generate an attribution map based on outputs of a first classification model and second classification model described in various exemplary embodiments. For example, a 4-cut model output and a 16-cut model output can be used to generate an attribution map. The inventors recognise that an attribution map points out or indicates important regions that contribute to a deep learning model's decision making. It is recognised that attribution analysis methods have been used in computer vision fields, such as Gradient-weighted Class Activation Mapping (Grad-CAM), integrated gradients (IG), and occlusion maps. These methods have also been used in deep learning based medical imaging processing, usually as cross-references with manual labelled features. However, the inventors recognise that most of these methods require tracing back to the input space level, such as usage of IG and occlusion maps, which is time-consuming (comparing with model prediction). For other methods such as Grad-CAM, the inventors recognise that such methods also need to trace back to the last convolutional layer of the deep learning model.

In contrast to the above-mentioned methods, in the exemplary embodiment, the 4-cut and 16-cut model outputs naturally form a regional attribution map. Therefore, in the description herein, it is referred to as a self-embedded attribution map. In the exemplary embodiment, a heatmap mask is generated based on the self-embedded attribution map to visualise the significant regions (e.g., regions showing highly probable BCC lesions).

In the exemplary embodiment, for better visualisation, the 4-cut model outputs O₄ and 16-cut model outputs O₁₆ are superimposed under the weights of 0.250₄+0.750₁₆ and Min-Max normalised.

In FIG. 6, the set 600 of heatmaps of the self-embedded attribution map is shown.

In the exemplary embodiment, the self-embedded attribution heatmaps are generated based on the NSC dataset. For better visualisation, a customized HSV (Hue Saturation Value) colour mapping is used instead of Jet colour mapping, and the heatmaps are generated by ‘colouration’ instead of simply superimposing a colour mask. The customised HSV colour map is set from blue (0, 0, 255) to red (255, 0, 0), and the value or the lightness of each point in the HSV colour map is constant. Through ‘colouration’, each grayscale pixel in original RCM images is changed to a coloured pixel by multiplying the corresponding hue value by its own grayscale pixel value. In the exemplary embodiment, such heatmap visualisation can preserve the original lightness information provided by the original RCM images to a significant extent, so that healthcare professionals (e.g., dermatologists) can better investigate the BCC lesions on the generated heatmaps. In the coloured heatmaps of FIG. 6, BCC attribution is shown across a range of colours. For a heatmap value of 0, the colour is blue with RGB value=(0, 0, 255) and for a heatmap value of 1, the colour is red with RGB value=(255, 0, 0). From the heatmap values between 0 to 1, the hue angle changes from 0° to 240° with full saturation. For example, the colour gradient ranges from blue to turquoise to green to yellow to red. In the set 600, originally provided with colour, the heatmaps show a variety of colours with different colour gradients according to the BCC attribution scale described above. Thus, the set 600 of heatmaps may make it relatively easier for a user to identify more probable BCC features.

The inventors recognise that in general, the heated regions (>0.5 in BCC attribution) in self-embedded attribution covers 92.20% of Grad-CAM heated regions generated for the same images. The inventors recognise that although the visualisations are generated under different generation approaches, they share a high agreement in locating BCC lesions.

In the exemplary embodiment, using a self-embedded attribution map offers several useful features. Firstly, it is recognised that an attribution map is naturally embedded in the hierarchical ensemble structure of the exemplary embodiment. Therefore, the self-embedded attribution map can be directly generated during model prediction, e.g., without having to trace back to an input space. Secondly, within the hierarchical ensemble structure of the exemplary embodiment, a 1-cut, 4-cut, and 16-cut models (corresponding to a third classification model, a first classification model, and a second classification model described in various exemplary embodiments) may not be restricted to convolutional networks. In the exemplary embodiment, even when other types of classification models/techniques are used, the hierarchical ensemble structure may still be able to generate a self-embedded attribution map. Thirdly, the inventors recognise that a self-embedded heatmap can provide an acceptable level of accuracy in its results and also, does not provide differing results from other known visualisation techniques such as usage of Grad-CAM, IG, occlusion maps. It is recognised that it may be possible to apply those known attribution analysis methods on the 1-cut, 4-cut, and 16-cut models of exemplary embodiments, and together generate an attribution map with even higher resolution and precision.

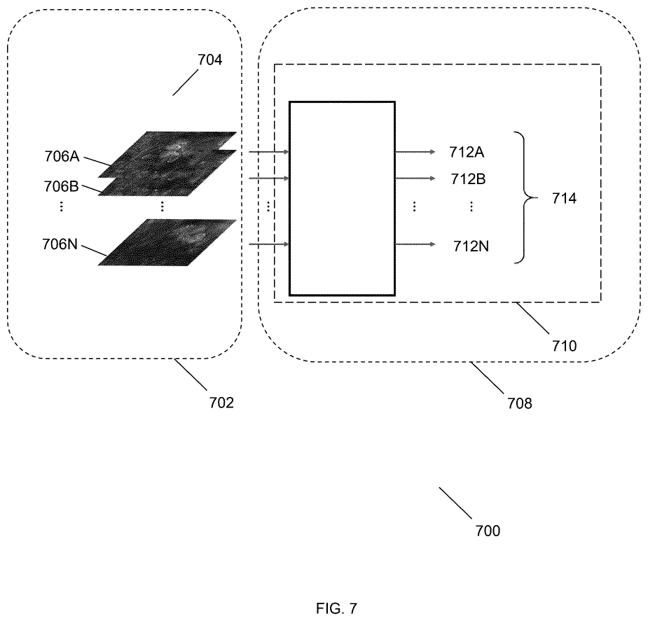

FIG. 7 is a schematic diagram of a system framework for classification of BCC in another exemplary embodiment. In this exemplary embodiment, the system 700 functions substantially similarly to the system 100 described with reference to FIG. 1. The system 700 for classification of BCC is based on confocal microscopy, e.g., reflectance confocal microscopy.

In the exemplary embodiment, the system 700 may receive input image sequence data from a confocal microscopy device stage 702 (compare confocal microscopy device 102 of FIG. 1). The confocal microscopy device stage 702 is arranged to obtain/capture and compile image sequence(s) (see example scanning sequence 704) with each image sequence comprising a plurality of images (see example images 706A, 706B, . . . 706N). In the image sequence (e.g., scanning sequence 704), each image (e.g., images 706A, 706B, . . . 706N) is an image of a skin surface of a subject obtained at a pre-determined depth with respect to a skin surface of a subject. The images e.g., 706A, 706B may be obtained at consecutive depths. The subject, for example, may be a human subject.

In the exemplary embodiment, the confocal microscopy device stage 702 comprises a confocal microscopy system/device VivaScope™ 3000 which is used for confocal image scanning. With the usage of the device, the confocal microscopy device stage 702 has a spatial resolution at cellular level of 1.2 μm. For vertical image scanning, the confocal microscopy device stage 702 has 5.0 μm vertical resolution, and the scanning speed is larger than 6 frames per second. A 30-frame sequence scanning can therefore be finished within 5 seconds. For a single frame, the field of view (FOV) is 750×750 μm², which the inventors recognise is sufficient to capture significant BCC features for classification. The penetration depth is 150 μm, which the inventors recognise is sufficient to reach the dermis of facial skin, which is typically the prevalent site of BCC. This depth is also close to stratum basale, where BCC typically originates. Therefore, in the exemplary embodiment, the confocal microscopy device stage 702 is recognised by the inventors to be useful in early detection of BCC.

In the exemplary embodiment, the system 700 comprises a processing module 708 (compare processing module 104 of FIG. 1) arranged to receive, from the confocal microscopy device 702, an input image sequence 704. The processing module 708 may be coupled to an input member (not shown) that can receive the input image sequence 704 and that is arranged to input the input image sequence 704 comprising the plurality of images 706A, 706B, . . . 706N to the processing module 708.

In the exemplary embodiment, the system 700 further comprises a prediction module 710 (compare prediction module 200 of FIG. 2) coupled to the processing module 708. In some exemplary embodiments, the processing module 708 can be the prediction module 710, i.e., it implements the functions of the prediction module 710. The prediction module 710 comprises a hierarchical ensemble structure of a plurality of classification models. In the exemplary embodiment, the hierarchical ensemble structure may have a first classification model, a second classification model, and a third classification model. The plurality of classification models are trained before being used in the hierarchical ensemble structure (e.g., see an exemplary method of training models described with reference to FIG. 5A). In the exemplary embodiment, the operations of the prediction module 710 may be instructed by the processing module 708.

In the exemplary embodiment, the system 700 further comprises a storage medium (not shown) coupled to the processing module 708. The storage medium may store instructions/code that are executable by the processing module 708. In some exemplary embodiments, the processing module 708 may be configured to retrieve and execute the instructions from the storage medium, and to provide the prediction module 710 (and/or the functions of the prediction module 710).

In the exemplary embodiment, the storage medium may further comprise a deep learning database (not shown). The deep learning database stores a plurality of classification models (e.g., a first classification model, a second classification model, and a third classification model) available for retrieval to assemble the hierarchical ensemble structure of the prediction module 710 e.g., for BCC classification. In the exemplary embodiment, the processing module 708 is configured to retrieve the plurality of classification models to assemble the hierarchical ensemble structure.

In the exemplary embodiment, the processing module 708 is configured to process each image of an input image sequence (e.g., images 706A, 706B, . . . 706N of input image sequence 704) as a whole image to obtain a plurality of first sub-sections and to further process each first sub-section to obtain a corresponding plurality of second sub-sections.

In the exemplary embodiment, the processing module 708 is configured to transmit each first sub-section to a first classification model of the prediction module 710 trained to process the first sub-sections and to transmit each of the corresponding plurality of second sub-sections to a second classification model of the prediction module 710 trained to process the second sub-sections. The processing module 708 is also configured to transmit the whole image (e.g., images 706A, 706B, . . . 706N of input image sequence 704) to a third classification model of the prediction module 710 trained to process the whole image.

In the exemplary embodiment, the prediction module 710 is configured to provide a respective block prediction result corresponding to each first sub-section. The respective block prediction result is based on respective prediction results of the first classification model for each first sub-section and the second classification model for the corresponding plurality of second sub-sections (i.e., corresponding to the first sub-section).

In the exemplary embodiment, the prediction module 710 is configured to provide a preliminary whole image prediction result for each image (e.g., images 706A, 706B, . . . 706N of input image sequence 704) based on the third classification model.

In the exemplary embodiment, the prediction module 710 is configured to determine a probability of BCC of each image of the input image sequence (e.g., probability of BCC 712A for image 706A, probability of BCC 712B for image 706B, until probability of BCC 712N for image 706N) based on the preliminary whole image prediction result of the each image and a plurality of the respective block prediction results corresponding to the first sub-sections of the each image. In the exemplary embodiment, the probability of BCC for each image (e.g., probability of BCC 712A, 712B, . . . 712N) may provide an indication as to the extent to which each image of the input image sequence (e.g., images 706A, 706B, . . . 706N of input image sequence 704) shows BCC features (e.g., BCC lesions).

In the exemplary embodiment, the processing module 708 is configured to determine a BCC classification 714 of the input image sequence (e.g., a final prediction/score) based on the determined probability of BCC of each image of the input image sequence (e.g., probability of BCC 712A, 712B, . . . 712N for images 706A, 706B, . . . 706N respectively).

In the exemplary embodiment, for generating a probability of BCC for each image of the input image sequence (e.g., probability of BCC 712A, 712B, . . . 712N), the processing module 708 may be configured to rely on a framework substantially similar to the same described with reference to FIG. 3. Further, for generating a BCC classification 714, the processing module 708 may be configured to implement a method of generating a BCC classification that is substantially similar to a method described with reference to FIGS. 4A and 4B.

In the exemplary embodiment, the system 700 further comprises an output device (not shown) coupled to the processing module 708. In the exemplary embodiment, the processing module 708 is configured to transmit the determined BCC classification 714 of the input image sequence 704 to the output device. In the exemplary embodiment, the output device is configured to provide/present the determined BCC classification 714 of the input image sequence 704 to a user. In the exemplary embodiment, the output device may be provided in the form of a monitor or a display that displays a user interface (e.g., a graphical user interface).

In the exemplary embodiment, the processing module 708 may be configured to inform a user of the determined BCC classification 714 of the input image sequence 704 via the output device. For example, if the BCC classification 714 (e.g., a final prediction/classification) of the input image sequence 704 is, for example, 0.8 (an exemplary value), the processing module 708 is configured to inform the user that the input image sequence 704 has a BCC prediction score of 0.8 and is classified as having BCC or showing a significant probability of BCC.

In the exemplary embodiment, the processing module 708 may be further configured to present corresponding heatmaps indicating significant regions (e.g., regions showing highly probable BCC lesions) in an image to the user via the user interface. As an example, for each individual image that has a determined probability of BCC that is more than a pre-determined value (e.g., 0.5), when such an image is to be presented to the user via the user interface, the processing module 708 may be configured to generate heatmaps for sub-sections of the individual image and be presented to the user e.g., together with the individual image via the user interface. For example, the heatmaps may be generated based on outputs of the first classification model and of the second classification model for the individual image. The heatmaps may be generated substantially similarly to the method described with reference to FIG. 6 for example.

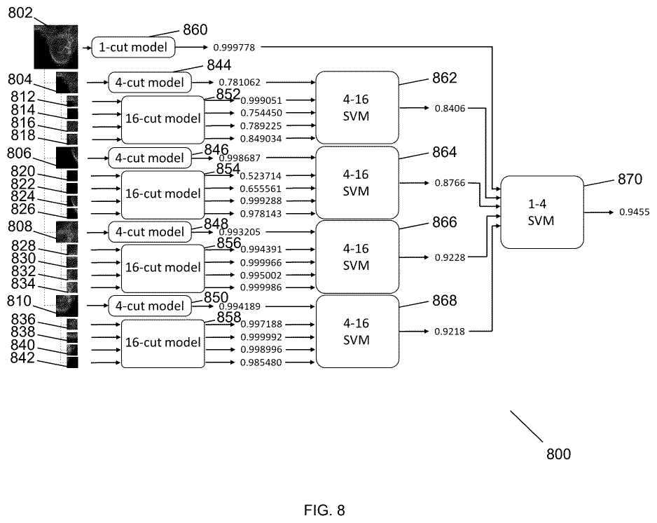

FIG. 8 is a schematic illustration of an example of generating a probability of BCC for an image of an input image sequence in another exemplary embodiment. The system 700 of FIG. 7 may be used for generating the probability of BCC for the image of the exemplary embodiment.

In the exemplary embodiment, a whole image 802 is processed by a hierarchical ensemble structure 800. The hierarchical ensemble structure 800 shown in FIG. 8 is similar to the framework 300 described with reference to FIG. 3 and may be implemented/performed by a processing module (e.g., processing module 708 described with reference to FIG. 7) and/or with the use of a prediction module (e.g., prediction module 710 described with reference to FIG. 7). For ease of reference, like naming conventions are used for exemplary implementations of similar modules as described in FIG. 7.

In the exemplary embodiment, the processing module processes the whole image 802 of an input image sequence (e.g., compare images 706A, 706B, . . . 706N of input image sequence 704 described with reference to FIG. 7) as a whole image to obtain a plurality of first sub-sections 804, 806, 808, and 810, and to further process each first sub-sections 804, 806, 808, and 810 to obtain a corresponding plurality of second sub-sections. Corresponding second sub-sections 812, 814, 816, and 818 are obtained from a first sub-section 804, corresponding second sub-sections 820, 822, 824, and 826 are obtained from a first sub-section 806, corresponding second sub-sections 828, 830, 832, and 834 are obtained from a first sub-section 808, and corresponding second sub-sections 836, 838, 840, and 842 are obtained from a first sub-section 810.

In the exemplary embodiment, the first sub-sections 804, 806, 808, and 810 are 4-cut images while the corresponding second sub-sections are 16-cut images (i.e. four 16-cut images being obtained from each 4-cut image).

In the exemplary embodiment, the processing module transmits each first sub-section 804, 806, 808, and 810 to a first classification model of the prediction module trained to process the first sub-sections (e.g. a 4-cut model labelled as 844, 846, 848, and 850 for four respective different blocks, each block corresponding to a first sub-section). The processing module also transmits each of the corresponding plurality of second sub-sections 812, 814, 816, 818, 820, 822, 824, 826, 828, 830, 832, 834, 836, 838, 840, 842 to a second classification model of the prediction module trained to process the second sub-sections (e.g. a 16-cut model labelled as 852, 854, 856, 858 for the four respective different blocks, each block corresponding to a first sub-section). The processing module further transmits the whole image 802 to a third classification model of the prediction module trained to process the whole image (e.g. a 1-cut model 860).

In the exemplary embodiment, the 4-cut model provides 4-cut prediction results, with one 4-cut prediction result generated for each of the first sub-sections 804, 806, 808, and 810. In FIG. 8, the 4-cut prediction result for the first sub-section 804 is 0.781062 (see output of numeral 844), the 4-cut prediction result for the first sub-section 806 is 0.998687 (see output of numeral 846), the 4-cut prediction result for the first sub-section 808 is 0.993205 (see output of numeral 848), and the 4-cut prediction result for the first sub-section 810 is 0.994189 (see output of numeral 850). The 16-cut model provides 16-cut prediction results, with one 16-cut prediction result generated for each of the corresponding plurality of second sub-sections 812, 814, 816, 818, 820, 822, 824, 826, 828, 830, 832, 834, 836, 838, 840, 842. For each block, the 16-cut prediction results are grouped with the corresponding 4-cut prediction result. For example, the prediction results for 16-cut images 812, 814, 816, 818 are grouped with their corresponding 4-cut image 804.

In FIG. 8, the 16-cut prediction results for the corresponding second sub-sections 812, 814, 816, and 818 (i.e. corresponding to the first sub-section 804) are 0.999051, 0.754450, 0.789225, and 0.849034 respectively (see output of numeral 852). The 16-cut prediction results for the corresponding second sub-sections 820, 822, 824, and 826 (i.e. corresponding to the first sub-section 806) are 0.523714, 0.655561, 0.999288, and 0.978143 respectively (see output of numeral 854). The 16-cut prediction results for the corresponding second sub-sections 828, 830, 832, and 834 (i.e. corresponding to the first sub-section 808) are 0.994391, 0.999966, 0.995002, and 0.999986 respectively (see output of numeral 856). The 16-cut prediction results for the corresponding second sub-sections 836, 838, 840, and 842 (i.e. corresponding to the first sub-section 810) are 0.997188, 0.999992, 0.998996, and 0.985480 (see output of numeral 858) respectively.

In the exemplary embodiment, the prediction module further provides a respective block prediction result corresponding to each first sub-section 804, 806, 808, and 810 by using a block SVM (e.g. a trained 4-16 SVM labelled as 862, 864, 866, and 868 for four respective different blocks, each block corresponding to a first sub-section). To obtain each block prediction result, the respective 4-cut prediction result of the first classification model for the corresponding first sub-section e.g. 804, 806, 808, and 810 and the respective corresponding 16-cut prediction results of the second classification model for the corresponding plurality of second sub-sections e.g. 812, 814, 816, 818, 820, 822, 824, 826, 828, 830, 832, 834, 836, 838, 840, 842 are transmitted to the 4-16 SVM e.g. at numerals 862, 864, 866, and 868. For example, the 4-cut model prediction result at numeral 844 and the 16-cut prediction results at numeral 852 are transmitted to the 4-16 SVM at numeral 862. In the exemplary embodiment, the respective block prediction results are provided by the block SVM (e.g. the 4-16 SVM labelled as 862, 864, 866, and 868). In FIG. 8, the block prediction result provided by 4-16 SVM at numeral 862 is 0.8406, the block prediction result provided by 4-16 SVM at numeral 864 is 0.8766, the block prediction result provided by 4-16 SVM at numeral 866 is 0.9228, and the block prediction result provided by 4-16 SVM at numeral 868 is 0.9218.

In the exemplary embodiment, the prediction module also provides a preliminary whole image prediction result for the whole image 802 based on the third classification model (e.g. a 1-cut model 860). In FIG. 8, the preliminary whole image prediction result for the whole image 802 is 0.999778.

In the exemplary embodiment, the prediction module determines a probability of BCC of the whole image 802 based on the preliminary whole image prediction result of the whole image 802 (based on the third classification model (1-cut model 860) prediction result) and a plurality of the respective block prediction results corresponding to the first sub-sections 804, 806, 808, and 810 (i.e. the block prediction results provided by the block SVM (or the 4-16 SVM prediction results at numerals 862, 864, 866, and 868)). The prediction results of the 1-cut model 860 and the block prediction results (at numerals 862, 864, 866, and 868) are transmitted to an image-wise stage SVM (e.g. a trained 1-4 SVM 870). In the exemplary embodiment, the probability of BCC of the image 802 is provided by the image-wise stage SVM (the 1-4 SVM 870). In FIG. 8, the probability of BCC of the whole image 802 provided by 1-4 SVM 870 is 0.9455.

In the exemplary embodiment, the hierarchical ensemble structure 800 is used to process each image in an input image sequence (e.g., compare images 706A, 706B, . . . 706N of input image sequence 704 described with reference to FIG. 7), i.e. to obtain a respective probability of BCC of the each image.

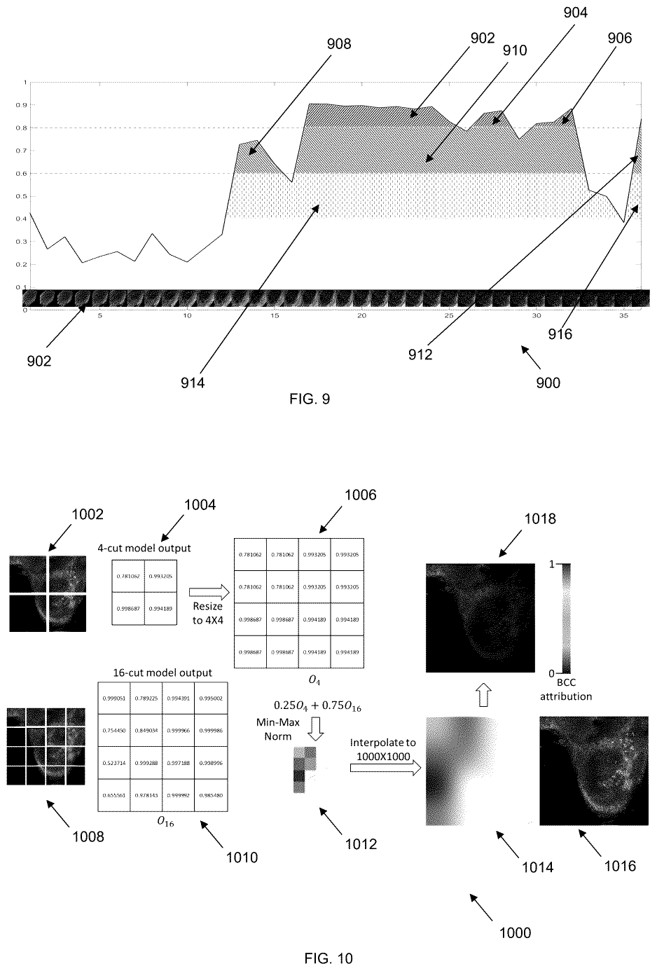

FIG. 9 is an example probability curve of an input image sequence in an exemplary embodiment. The probability curve shown in FIG. 9 is plotted based on the probability of BCC determined for each image in an input image sequence determined using the hierarchical ensemble structure 800 described with reference to FIG. 8 (see y-axis) against a relative depth of the input image sequence (see x-axis). For illustration purposes, a set of 36 images of the input image sequence are super-imposed on the x-axis (see numeral 902) and the probability of BCC determined for each image is plotted for the 36 images. It will be appreciated that the relative depth may be resized to any number of images.

In the exemplary embodiment, the plot 900 shows one or more areas of each consecutive part that are between BCC score values (or probabilities of BCC) of 0.8 to 1 (see e.g. reference numerals 902, 904, 906); between BCC score values 0.6 to 0.8 (see e.g. reference numerals 908, 910, 912); and between BCC score values 0.4 to 0.6 (see e.g. reference numerals 914, 916).

In the exemplary embodiment, a prediction module (e.g., prediction module 710 described with reference to FIG. 7) determines a BCC classification of the input image sequence based on the determined probability of BCC of each image of the input image sequence that is shown in FIG. 9. The determination of BCC classification is performed using a method that is substantially identical to the method described with reference to FIGS. 4A and 4B. That is, BCC classification is determined by calculating a sequence-wise BCC prediction score (B) using the following equation:

$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} + θ ) .$$

In the above equation, Sₐᵢ stands for the area of each consecutive part between BCC score values 0.8 to 1 (see reference numerals 902, 904, 906 of FIG. 9), and similarly, S_{bj} and S_{ck} stand for areas of each consecutive part between score values 0.6 to 0.8 (see reference numerals 908, 910, 912 of FIG. 9) and score values 0.4 to 0.6 (see reference numerals 914, 916 of FIG. 9) respectively. lₐᵢ, l_{bj} and l_{ck} stand for the corresponding length of Sₐᵢ, S_{bj}, and S_{ck} The respective lengths in the exemplary embodiment may be determined based on the relative depth (or the numbering of the each image of the input image sequence). A positive small value constant θ=0.01 is added to the equation to prevent a case of having B=ln 0.

In the exemplary embodiment, the sequence-wise BCC prediction score (B) may be informed to a user via an output device (compare output device of FIG. 7), e.g. through a user interface displayed at the output device. Further, the processing module may also generate one or more heatmaps for one or more images of the input image sequence and the processing module may transmit the heatmaps for display at the output device, e.g. through a user interface displayed at the output device.

FIG. 10 is a schematic flow diagram for illustrating generation of an example visual heatmap in an exemplary embodiment. In the exemplary embodiment, the flow diagram 1000 is based on the whole image 802 processed by the hierarchical ensemble structure 800 described with reference to FIG. 8, and the exemplary values shown in FIG. 10 are from FIG. 8. The heatmap generation is similar to the method for generating heatmaps described with reference to FIG. 6 and may be implemented/performed by a processing module (e.g., processing module 708 described with reference to FIG. 7). For ease of reference, like naming conventions are used for exemplary implementations of similar modules as described in FIG. 7.

In the exemplary embodiment, the processing module obtains and arranges 4-cut model outputs for four first sub-sections 1002 (obtained from a whole image of an input image sequence, compare the whole image 802 of FIG. 8) in a 2×2 grid 1004. In the grid 1004, the 4-cut model outputs are arranged such that the positions of each of the 4-cut model outputs correspond to the portion of the whole image that each 4-cut model output is based on. For example, the 4-cut model output value 0.781062 (compare output of numeral 844 of FIG. 8) positioned at the top left of the grid 1004 corresponds to the up-left (UL) portion of the whole image (i.e., the up-left first sub-section). This applies similarly to the other 4-cut model output values 0.998687 (in the bottom left of the grid for BL; compare output of numeral 846 of FIG. 8), 0.993205 (in the top right of the grid for UR; compare output of numeral 848 of FIG. 8), and 0.994189 (in the bottom right of the grid for BR; compare output of numeral 850 of FIG. 8) respectively.

In the exemplary embodiment, the processing module resizes each grid of the 2×2 grid to a 2×2 grid having the same output values of the each grid, thus resulting in a 4×4 grid 1006. The 4-cut model outputs (O₄) in the 4×4 grid 1006 are arranged such that the 4-cut model output values in the top left portion of the 4×4 grid 1006 (the top left 2×2 subgrids each showing the values 0.781062) correspond to the 4-cut model output value in the top left of the 2×2 grid 1004 (showing the value 0.781062). This applies similarly to the 4-cut model output values in the bottom left portion of the 4×4 grid 1006 (the bottom left 2×2 subgrids each showing the value 0.998687), the top right portion of the 4×4 grid 1006 (the top right 2×2 subgrids each showing the value 0.993205), and the bottom right portion of the 4×4 grid 1006 (the bottom right 2×2 subgrids each showing the value 0.994189).

In the exemplary embodiment, the processing module also obtains and arranges the 16-cut model outputs (O₁₆) for the sixteen second sub-sections 1008 (obtained from the same whole image, compare the whole image 802 of FIG. 8) in a 4×4 grid 1010. The 16-cut model outputs (O₁₆) are arranged such that the positions of each of the 16-cut model outputs correspond to the portion of the whole image that each 16-cut model output is based on, or correspond to the portion of its corresponding first sub-section that the 16-cut model output is based on. For example, in terms of 2×2 subgrids, the upper-left (UL) 2×2 subgrid of the 4×4 grid 1010 contain the 16-cut model output values 0.999051, 0.754450, 0.789225, and 0.849034 (compare output of numeral 852 of FIG. 8 and the output values corresponding to second sub-sections 812, 814, 816, and 818 of FIG. 8 which are the UL, BL, UR, BR of the first sub-section 808 of FIG. 8). These 16-cut model output values 0.999051, 0.754450, 0.789225, and 0.849034 are related to the upper-left grid of the 2×2 grid 1004 (of 4-cut model output value 0.781062) and the upper-left 2×2 subgrid of the 4×4 grid 1006. In terms of 2×2 subgrids of the 4×4 grid 1010, the above arrangement applies similarly to the other 16-cut model output values in the grid 1010.

In the exemplary embodiment, with the two 4×4 grids 1006, 1010 of the 4-cut model output results/values 04 and of the 16-cut model results/values 016 respectively, the processing module superimposes the two 4×4 grids 1006, 1010 under the weights of 0.25O₄+0.75O₁₆ to obtain a resultant 4×4 grid and performs an operation for Min-Max normalisation (e.g. for scaling data between 0 to 1 based on the minimum and maximum values present). It will be appreciated that in exemplary embodiments, the weights may be varied, i.e., other suitable weightage may be used. In the exemplary embodiment, the weightage given to O₁₆ is higher than that given to O₄ with the recognition that there is lesser information loss with the resizing of the 16-cut data as compared to the 4-cut data. The resultant 4×4 grid in terms of value converted to grayscale pixel value is shown as pixel value grid 1012. In terms of grayscale pixel value, the higher a value (i.e. closer to 1), the lighter is the grayscale colour (i.e. closer to white).

In the exemplary embodiment, the processing module applies interpolation (e.g., cubic spline interpolation) to the pixel value grid 1012 to obtain a heatmap mask 1014 of size 1000×1000 pixels, i.e., a size that corresponds to the whole image. The processing module then combines the heatmap mask 1014 with the whole image 1016. The processing module also provides colour transformation to the combination of the heatmap mask 1014 with the whole image 1016 to generate a visual heatmap 1018.

In the exemplary embodiment, for the combination and colour transformation, a customized HSV (Hue Saturation Value) colour mapping is used wherein the customised HSV colour map is set from blue (0, 0, 255) to red (255, 0, 0), and colour transformation is provided by ‘colouration’ wherein each grayscale pixel in the whole image is changed to a coloured pixel by multiplying the corresponding hue value by its own grayscale pixel value (determined by the heatmap mask 1014). In the exemplary embodiment, such heatmap visualisation can preserve the original lightness/intensity information provided by an original whole image to a significant extent, so that healthcare professionals (e.g., dermatologists) can better investigate the BCC lesions on the generated visual heatmap. In the visual heatmap 1018, BCC attribution is shown across a range of colours. For a heatmap value of 0, the colour is blue with RGB value=(0, 0, 255) and for a heatmap value of 1, the colour is red with RGB value=(255, 0, 0). From the heatmap values between 0 to 1, the hue angle changes from 0° to 240° with full saturation. For example, the colour gradient ranges from blue to turquoise to green to yellow to red. In the visual heatmap 1018, originally provided with colour, the visual heatmap 1018 shows a variety of colours with different colour gradients according to the BCC attribution scale described above. Thus, the visual heatmap 1018 may make it relatively easier for a user to identify more probable BCC features.

In the exemplary embodiment, the flow diagram 1000 thus illustrates a method which utilises the first classification model (e.g. 4-cut model) and the second classification model (e.g. 16-cut model) prediction results/output values (e.g., obtained from the example described with reference to FIG. 8) that naturally form a regional self-embedded attribution map.

In an exemplary embodiment, similar to the above embodiments, a method for classifying BCC may be provided that uses confocal microscopy images with deep learning techniques. In the exemplary embodiment, the method comprises the following steps that may be implemented with, for example, the system 700 of FIG. 7.

In the exemplary embodiment, a confocal microscopy system (e.g., confocal microscopy device stage 702 of FIG. 7) is used for image scanning. For example, the confocal microscopy system may be used for capturing and compiling confocal scanning image sequences, each image sequence comprising a plurality of images. In the exemplary embodiment, the confocal microscopy system may have a spatial resolution at cellular level of 1.25 μm. For vertical image scanning, the confocal microscopy system may have 5.0 μm vertical resolution, and the scanning speed may be larger than 6 frames per second. Further, the confocal microscopy system may be configured so that a 30-frame sequence scanning can be finished within 5 seconds. For a single frame, the field of view (FOV) may be 750×75 μm², which may be sufficient to capture significant BCC features for classification.

In the exemplary embodiment, a processing module (e.g., processing module 708 of FIG. 7) may create a database to store confocal microscopy scanning images of BCC subjects/patients and of normal subjects. Thus, the database stores at least two training groups of images. For example, the database stores a BCC training group of images and a non-BCC training group of images. The confocal microscopy scanning images of BCC subjects/patients may undergo data augmentation. In the exemplary embodiment, the BCC training group of images comprises images of known BCC subjects arranged as one or more BCC image sequences and the non-BCC training group of images comprises images of normal subjects (or non-BCC subjects or normal healthcare patients) arranged as one or more non-BCC image sequences.

In the exemplary embodiment, the processing module may group the augmented images of BCC subjects in a same sequence as the original images of BCC subjects for a training dataset.

In the exemplary embodiment, a matured image classification model of ResNet 101 is used. The depth information provided by confocal microscopy may be exploited e.g., by applying a 10-fold cross-validation split on the dataset. In the exemplary embodiment, the dataset is evenly divided into 10 folds, in which 8 folds are used for model training, and the other 2 folds are used as a validation set and a testing set. At least a first classification model for processing first sub-sections of an image (e.g., 4-cut model for processing 4-cut data of a whole image), a second classification model for processing second sub-sections from each first sub-section (e.g., 16-cut model for processing 16-cut data of a 4-cut segment) and a third classification model for processing a whole image (e.g., 1-cut model for processing a whole image) are trained, tested and validated for selection to assemble into a hierarchical ensemble structure. Other classification models such as a block SVM and an image-wise stage SVM may also be trained for assembly into the hierarchical ensemble structure.

In the exemplary embodiment, the method comprises predicting BCC using input confocal scanning image sequences by generating probabilities of BCC of each image within each input image sequence (from the input confocal scanning image sequences) based on the plurality of trained classification models of the hierarchical ensemble structure, and determining a BCC classification of an entire input image sequence based on the probability of BCC of each image of the input image sequence.

In the described exemplary embodiments, by making use of the high spatial resolution and depth information that can be provided in confocal scanning sequences (captured and compiled by a confocal microscopy device), in one or more experiments, the inventors are/were able to achieve experimental results of a sequential BCC classification accuracy of close to 100% in a 10-fold cross-validation evaluation. It is recognised that the performance is better than other BCC classification methods that the inventors are aware of.

In the described exemplary embodiments, with the provision of a confocal microscopy system for use with a system for classifying BCC (e.g., confocal microscopy device 102 of FIG. 1 and confocal microscopy device stage 702 of FIG. 7), it is possible to implement a method for classifying BCC that is non-invasive to subjects as compared to other methods for clinical diagnosis, such as histopathology. This may in turn allow a wide-range and regular examination of BCC.

Further, in the described exemplary embodiments, with usage of a confocal microscopy device which can capture BCC morphology with high spatial resolution and provide more comprehensive morphological information along depth, and with a confocal microscopy-based BCC classification method which uses deep learning, as well as a hierarchical ensemble model that maximises the utilisation of the information, the system for BCC classification can provide desirable performance (e.g., usefully output a BCC classification for an input image sequence) and make a non-invasive, quick, automatic and highly accurate BCC detection method possible. In the described exemplary embodiments, with usage of a confocal microscopy device, early detection and diagnosis of BCC may also be made possible.

In the described exemplary embodiments, the system for classifying BCC may allow a relatively quicker and faster detection of BCC when compared to performing manual examinations, and further enhance the efficiency in BCC diagnosis. For example, the system can be configured to complete sequential processing of input image sequences in a short span of time, e.g., within 1 minute, which is much faster when compared to other methods for diagnosing BCC, e.g., performing manual examination.

In the described exemplary embodiments, the output device may be in the form of a user interface. Providing a user interface may be useful in providing quick automatic diagnostic suggestions of BCC to users (e.g., human healthcare professionals and trained personnel, such as dermatologists).

In the described exemplary embodiments, the system for classifying BCC may usefully address an issue/problem of limited numbers of human healthcare professionals and trained personnel (e.g., dermatologists) that are capable of diagnosing BCC through confocal microscopy scanning results, and also an issue/problem of requiring a large amount of time and money in training such trained personnel. That is, with the described exemplary embodiments, the system for classifying BCC may address the above issues and significantly improve the efficiency in BCC diagnosis.

In the described exemplary embodiments, deep learning techniques are applied to BCC classification based on confocal microscopy. A database of confocal microscopy scanning images of BCC subjects and normal subjects is created e.g., for model training and future research and development. To fully exploit the depth information provided by confocal microscopy, BCC classification/prediction is determined based on a unit of confocal scanning sequence (or image sequence) instead of a single image (e.g., such as when using a single-image input for histopathology). Determining the BCC classification based on a unit of confocal scanning sequence (or image sequence) may usefully improve the accuracy of the determined BCC classification.

In the described exemplary embodiments, the system for BCC classification is configured to allow significant BCC features within confocal scanning images that are deterministic to a deep learning model's determination of the BCC classification (or prediction making) to be extracted and visualised, and may, if desired, be further cross-referenced with features in clinical diagnosis by human healthcare professionals and trained personnel (e.g., experienced dermatologists). The additional step of cross-referencing may usefully aid in improving the accuracy of the BCC classification outputted by the system.

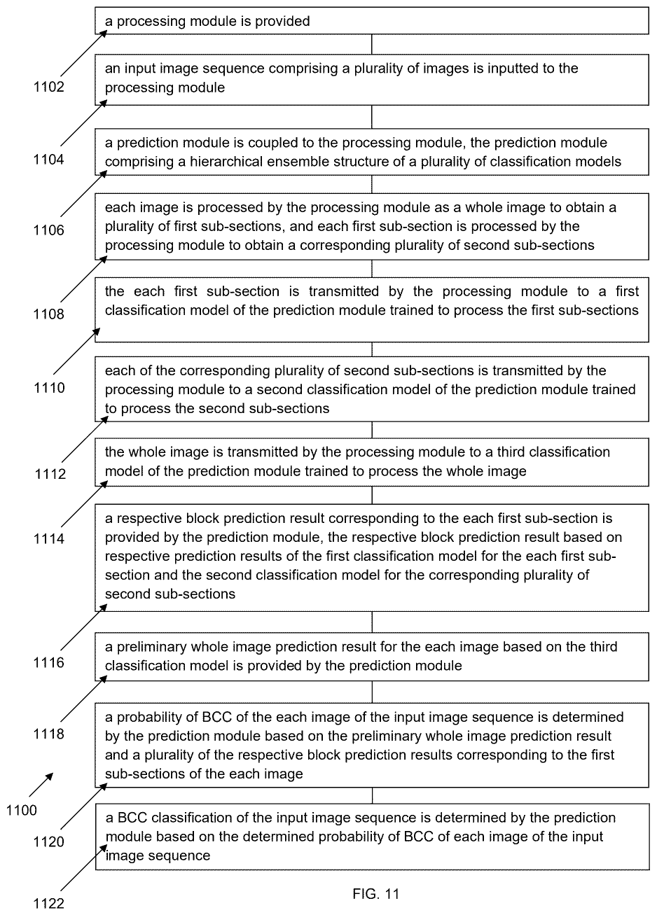

FIG. 11 is a schematic flowchart 1100 illustrating a method of classifying Basal Cell Carcinoma (BCC) in an exemplary embodiment. One or more steps of the method are computer-implemented. Alternatively, the method is a computer-implemented method.

At step 1102, a processing module is provided. At step 1104, an input image sequence comprising a plurality of images is inputted to the processing module. At step 1106, a prediction module is coupled to the processing module, the prediction module comprising a hierarchical ensemble structure of a plurality of classification models. At step 1108, each image is processed by the processing module as a whole image to obtain a plurality of first sub-sections, and each first sub-section is further processed by the processing module to obtain a corresponding plurality of second sub-sections. At step 1110, the each first sub-section is transmitted by the processing module to a first classification model of the prediction module trained to process the first sub-sections. At step 1112, each of the corresponding plurality of second sub-sections is transmitted by the processing module to a second classification model of the prediction module trained to process the second sub-sections. At step 1114, the whole image is transmitted by the processing module to a third classification model of the prediction module trained to process the whole image. At step 1116, a respective block prediction result corresponding to the each first sub-section is provided by the prediction module, the respective block prediction result based on respective prediction results of the first classification model for the each first sub-section and the second classification model for the corresponding plurality of second sub-sections. At step 1118, a preliminary whole image prediction result for the each image based on the third classification model is provided by the prediction module. At step 1120, a probability of BCC of the each image of the input image sequence is determined by the prediction module based on the preliminary whole image prediction result and a plurality of the respective block prediction results corresponding to the first sub-sections of the each image. At step 1122, a BCC classification of the input image sequence is determined by the prediction module based on the determined probability of BCC of each image of the input image sequence.

In another exemplary embodiment, there is provided a non-transitory tangible computer readable storage medium having stored thereon software instructions that, when executed by a processing module of a system for classification of basal cell carcinoma (BCC), cause the processing module to perform a method of classifying basal cell carcinoma (BCC), by executing the steps comprising providing a processing module; inputting an input image sequence comprising a plurality of images to the processing module; coupling a prediction module to the processing module, the prediction module comprising a hierarchical ensemble structure of a plurality of classification models; processing each image, by the processing module, as a whole image to obtain a plurality of first sub-sections and further processing, by the processing module, each first sub-section to obtain a corresponding plurality of second sub-sections; transmitting, by the processing module, the each first sub-section to a first classification model of the prediction module trained to process the first sub-sections; transmitting, by the processing module, each of the corresponding plurality of second sub-sections to a second classification model of the prediction module trained to process the second sub-sections; transmitting, by the processing module, the whole image to a third classification model of the prediction module trained to process the whole image; providing, by the prediction module, a respective block prediction result corresponding to the each first sub-section, the respective block prediction result based on respective prediction results of the first classification model for the each first sub-section and the second classification model for the corresponding plurality of second sub-sections; providing, by the prediction module, a preliminary whole image prediction result for the each image based on the third classification model; determining, by the prediction module, a probability of BCC of the each image of the input image sequence based on the preliminary whole image prediction result and a plurality of the respective block prediction results corresponding to the first sub-sections of the each image; and determining, by the prediction module, a BCC classification of the input image sequence based on the determined probability of BCC of each image of the input image sequence.

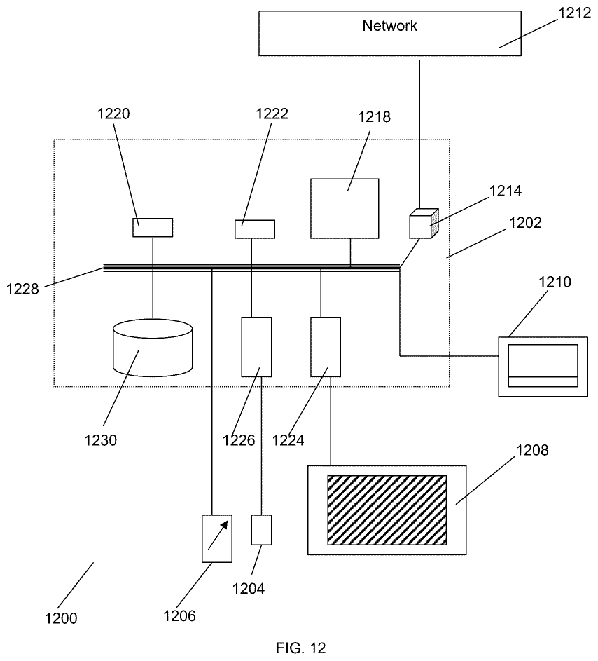

Different exemplary embodiments can be implemented in the context of data structure, program modules, program and computer instructions executed in a computer implemented environment. A general purpose computing environment is briefly disclosed herein. One or more exemplary embodiments may be embodied in one or more computer systems, such as is schematically illustrated in FIG. 12.

One or more exemplary embodiments may be implemented as software, such as a computer program being executed within a computer system 1200, and instructing the computer system 1200 to conduct a method of an exemplary embodiment.

The computer system 1200 comprises a computer unit 1202, input modules such as a keyboard 1204 and a pointing device 1206 and a plurality of output devices such as a display 1208, and printer 1210. A user can interact with the computer unit 1202 using the above devices. The pointing device can be implemented with a mouse, track ball, pen device or any similar device. One or more other input devices (not shown) such as a joystick, game pad, satellite dish, scanner, touch sensitive screen or the like can also be connected to the computer unit 1202. The display 1208 may include a cathode ray tube (CRT), liquid crystal display (LCD), field emission display (FED), plasma display or any other device that produces an image that is viewable by the user.

The computer unit 1202 can be connected to a computer network 1212 via a suitable transceiver device 1214, to enable access to e.g., the Internet or other network systems such as Local Area Network (LAN) or Wide Area Network (WAN) or a personal network. The network 1212 can comprise a server, a router, a network personal computer, a peer device or other common network node, a wireless telephone or wireless personal digital assistant. Networking environments may be found in offices, enterprise-wide computer networks and home computer systems etc. The transceiver device 1214 can be a modem/router unit located within or external to the computer unit 1202, and may be any type of modem/router such as a cable modem or a satellite modem. As an example, the transceiver device 1214 may be an input member to receive image sequences to input to a processing module of described exemplary embodiments.

It will be appreciated that network connections shown are exemplary and other ways of establishing a communications link between computers can be used. The existence of any of various protocols, such as TCP/IP, Frame Relay, Ethernet, FTP, HTTP and the like, is presumed, and the computer unit 1202 can be operated in a client-server configuration to permit a user to retrieve web pages from a web-based server. Furthermore, any of various web browsers can be used to display and manipulate data on web pages.

The computer unit 1202 in the example comprises a processor 1218, a Random Access Memory (RAM) 1220 and a Read Only Memory (ROM) 1222. The ROM 1222 can be a system memory storing basic input/output system (BIOS) information. The RAM 1220 can store one or more program modules such as operating systems, application programs and program data.

The processor 1218 may be a processing module of described exemplary embodiments. For example, compare the processing module 104 of FIG. 1 and the processing module 708 of FIG. 7. The processor 1218 may be configured to instruct the operations of a prediction module (e.g., prediction module 200 of FIG. 2). The processor 1218 may alternatively be a prediction module (e.g., prediction module 200 of FIG. 2), i.e., the processor 1218 implements the functions of the prediction module. The RAM 1220 and/or the ROM 1222 may be used to store one or more deep learning databases. In some exemplary embodiments, the RAM 1220 and/or the ROM 1222 may store instructions/code executable by the processor 1218 e.g. to implement the functions of the prediction module, to provide the prediction module, to call up the prediction module etc.

The computer unit 1202 further comprises a number of Input/Output (I/O) interface units, for example I/O interface unit 1224 to the display 1208, and I/O interface unit 1226 to the keyboard 1204. The components of the computer unit 1202 typically communicate and interface/couple connectedly via an interconnected system bus 1228 and in a manner known to the person skilled in the relevant art. The bus 1228 can be any of several types of bus structures including a memory bus or memory controller, a peripheral bus, and a local bus using any of a variety of bus architectures.

It will be appreciated that other devices can also be connected to the system bus 1228. For example, a universal serial bus (USB) interface can be used for coupling a video or digital camera to the system bus 1228. An IEEE 1394 interface may be used to couple additional devices to the computer unit 1202. Other manufacturer interfaces are also possible such as FireWire developed by Apple Computer and i.Link developed by Sony. Coupling of devices to the system bus 1228 can also be via a parallel port, a game port, a PCI board or any other interface used to couple an input device to a computer. It will also be appreciated that, while the components are not shown in the figure, sound/audio can be recorded and reproduced with a microphone and a speaker. A sound card may be used to couple a microphone and a speaker to the system bus 1228. It will be appreciated that several peripheral devices can be coupled to the system bus 1228 via alternative interfaces simultaneously.

The system bus 1228 may be used to couple, e.g. via a mating input member, to a removable storage device (e.g., a USB drive, an external hard drive) to receive one or more input image or input image sequences for input to the processor 1218.

An application program can be supplied to the user of the computer system 1200 being encoded/stored on a data storage medium such as a CD-ROM or flash memory carrier. The application program can be read using a corresponding data storage medium drive of a data storage device 1230. The data storage medium is not limited to being portable and can include instances of being embedded in the computer unit 1202. The data storage device 1230 can comprise a hard disk interface unit and/or a removable memory interface unit (both not shown in detail) respectively coupling a hard disk drive and/or a removable memory drive to the system bus 1228. This can enable reading/writing of data. Examples of removable memory drives include magnetic disk drives and optical disk drives. The drives and their associated computer-readable media, such as a floppy disk provide nonvolatile storage of computer readable instructions, data structures, program modules and other data for the computer unit 1202. It will be appreciated that the computer unit 1202 may include several of such drives. Furthermore, the computer unit 1202 may include drives for interfacing with other types of computer readable media.

The application program is read and controlled in its execution by the processor 1218. Intermediate storage of program data may be accomplished using RAM 1220. The method(s) of the exemplary embodiments can be implemented as computer readable instructions, computer executable components, or software modules. One or more software modules may alternatively be used. These can include an executable program, a data link library, a configuration file, a database, a graphical image, a binary data file, a text data file, an object file, a source code file, or the like. When one or more computer processors execute one or more of the software modules, the software modules interact to cause one or more computer systems to perform according to the teachings herein.

The operation of the computer unit 1202 can be controlled by a variety of different program modules. Examples of program modules are routines, programs, objects, components, data structures, libraries, etc. that perform particular tasks or implement particular abstract data types. The exemplary embodiments may also be practiced with other computer system configurations, including handheld devices, multiprocessor systems, microprocessor-based or programmable consumer electronics, network PCs, minicomputers, mainframe computers, personal digital assistants, mobile telephones and the like. Furthermore, the exemplary embodiments may also be practiced in distributed computing environments where tasks are performed by remote processing devices that are linked through a wireless or wired communications network. In a distributed computing environment, program modules may be located in both local and remote memory storage devices.

In the description herein, the terms “coupled” or “connected” as used are intended to cover both directly connected or connected through one or more intermediate means, unless otherwise stated. In some exemplary embodiments, the prediction module may be provided as a software module or function, and the processing module may be coupled to the prediction module by accessing or calling up the prediction module and/or the functions of the prediction module. In some exemplary embodiments, the processing module, in coupling to the software module or functions, may be the prediction module.

The use of “a”, “an” or “the” is intended to mean “one or more” unless it is described specifically to the contrary.

The terms “configured to (perform a task/action)”, “configured for (performing a task/action)” and the like as used in this description include being programmable, programmed, connectable, wired or otherwise constructed to have the ability to perform the task/action when arranged or installed as described herein. The terms “configured to (perform a task/action)”, “configured for (performing a task/action)” and the like are intended to cover “when in use, the task/action is performed”, e.g., specifically to and/or specifically configured to and/or specifically arranged to and/or specifically adapted to do or perform a task/action.

The term “and/or”, e.g., “X and/or Y” is understood to mean either “X and Y” or “X or Y” and should be taken to provide explicit support for both meanings or for either meaning. The use of “or” is intended to mean an “inclusive or,” and not an “exclusive or” unless it is described specifically to the contrary.

The terms “associated with”, “related to” and the like used herein when referring to two elements refers to a broad relationship between the two elements. The relationship includes, but is not limited to, a physical, a chemical or a biological relationship. For example, when element A is associated with element B, elements A and B may be directly or indirectly attached to each other or element A may contain element B or vice versa.

The terms “exemplary embodiment”, “example embodiment”, “exemplary implementation”, “exemplarily” and the like used herein are intended to indicate an example of matters described in the present disclosure. Such an example may relate to one or more features defined in the claims and is not necessarily intended to emphasise a best example or any essentialness of any features.

The description herein may be, in certain portions, explicitly or implicitly described as algorithms and/or functional operations that operate on data within a computer memory or an electronic circuit. These algorithmic descriptions and/or functional operations are usually used by those skilled in the information/data processing arts for efficient description. An algorithm is generally relating to a self-consistent sequence of steps leading to a desired result. The algorithmic steps can include physical manipulations of physical quantities, such as electrical, magnetic or optical signals capable of being stored, transmitted, transferred, combined, compared, and otherwise manipulated.

Further, unless specifically stated otherwise, and would ordinarily be apparent from the following, a person skilled in the art will appreciate that throughout the present specification, discussions utilizing terms such as “scanning”, “calculating”, “determining”, “replacing”, “generating”, “initializing”, “outputting”, and the like, refer to action and processes of an instructing processor/computer system, similar or electronic circuit/device/component, that manipulates/processes and transforms data represented as physical quantities within the described system into other data similarly represented as physical quantities within the system or other information storage, transmission or display devices etc.

The description also discloses relevant device/apparatus for performing the steps of the described methods. Such apparatus may be specifically constructed for the purposes of the methods, or may comprise a general purpose computer/processor or other device selectively activated or reconfigured by a computer program stored in a storage member. The algorithms and displays described herein are not inherently related to any particular computer or other apparatus. It is understood that general purpose devices/machines may be used in accordance with the teachings herein. Alternatively, the construction of a specialized device/apparatus to perform the method steps may be desired.

In addition, it is submitted that the description also implicitly covers a computer program, in that it would be clear that the steps of the methods described herein may be put into effect by computer code. It will be appreciated that a large variety of programming languages and coding can be used to implement the teachings of the description herein. Moreover, the computer program if applicable is not limited to any particular control flow and can use different control flows without departing from the scope of the invention.

Furthermore, one or more of the steps of the computer program if applicable may be performed in parallel and/or sequentially. Such a computer program if applicable may be stored on any computer readable medium. The computer readable medium may include storage devices such as magnetic or optical disks, memory chips, or other storage devices suitable for interfacing with a suitable reader/general purpose computer. In such instances, the computer readable storage medium is non-transitory. Such storage medium also covers all computer-readable media e.g., medium that stores data only for short periods of time and/or only in the presence of power, such as register memory, processor cache and Random Access Memory (RAM) and the like. The computer readable medium may even include a wired medium such as exemplified in the Internet system, or wireless medium such as exemplified in Bluetooth technology. The computer program when loaded and executed on a suitable reader effectively results in an apparatus that can implement the steps of the described methods.

The exemplary embodiments may also be implemented as hardware modules. A module is a functional hardware unit designed for use with other components or modules. For example, a module may be implemented using digital or discrete electronic components, or it can form a portion of an entire electronic circuit such as an Application Specific Integrated Circuit (ASIC). A person skilled in the art will understand that the exemplary embodiments can also be implemented as a combination of hardware and software modules.

Additionally, when describing some embodiments, the disclosure may have disclosed a method and/or process as a particular sequence of steps. However, unless otherwise required, it will be appreciated the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For an example, when “comprising” is used, reference to a “one” feature is also intended to be a reference to “at least one” of that feature. Terms such as “consisting”, “consist”, and the like, may, in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

In the description, a 10-fold cross validation split technique is generally used. It will be appreciated that the exemplary embodiments are not limited as such and any k-fold cross validation techniques can be used instead or in complement. For example, k can be an integer from 4 to 10.

In the description, it will be appreciated that usage of the phrase “deep learning” is understood to also mean similar concepts or fields such as machine learning, artificial intelligence, usage of neural networks etc.

In some described exemplary embodiments, a whole image obtained by a confocal microscopy device (e.g., a RCM image obtained by a RCM device) may be divided up to sixteen second sub-sections (from four first sub-sections). Utilising sixteen second sub-sections may usefully utilise the resolution provided by a RCM device in cases where the input size of a ResNet model is 224×224 pixels, and the image size of a RCM image is 1000×1000 pixels. It will be appreciated that the exemplary embodiments are not limited as such. That is, a whole image may be further divided (e.g., divided up to sixty-four sub-sections or more). For example, a 64-cut model may be trained to provide 64-cut prediction results and the trained 64-cut model may be used in the hierarchical ensemble structure. It will also be appreciated that if the source image resolution is larger, e.g., the source image size is 5000×5000 pixels, such further divisions (beyond sixteen sub-sections or more) may be beneficial.

In the described exemplary embodiments, the prediction module implements specific functions and is described as coupled to the processing module. It will be appreciated that the exemplary embodiments are not limited as such. That is, the functions of the prediction module may be implemented by the processing module, i.e., the processing module is also the prediction module. In such exemplary embodiments, the system for classification of BCC may comprise a computer readable medium coupled to the processing module, the computer-readable medium storing instructions executable by the processing module that when executed by the processing module, provide the functions of the prediction module. For example, the computer-readable medium may store instructions executable by the processing module that when executed by the processing module, provide a method of classifying basal cell carcinoma (BCC), the method comprising inputting an input image sequence comprising a plurality of images to the processing module; providing a hierarchical ensemble structure of a plurality of classification models; processing each image as a whole image to obtain a plurality of first sub-sections and further processing each first sub-section to obtain a corresponding plurality of second sub-sections; transmitting the each first sub-section to a first classification model of the hierarchical ensemble structure trained to process the first sub-sections; transmitting each of the corresponding plurality of second sub-sections to a second classification model of the hierarchical ensemble structure trained to process the second sub-sections; transmitting the whole image to a third classification model of the hierarchical ensemble structure trained to process the whole image; providing a respective block prediction result corresponding to the each first sub-section, the respective block prediction result based on respective prediction results of the first classification model for the each first sub-section and the second classification model for the corresponding plurality of second sub-sections; providing a preliminary whole image prediction result for the each image based on the third classification model; determining a probability of BCC of the each image of the input image sequence based on the preliminary whole image prediction result and a plurality of the respective block prediction results corresponding to the first sub-sections of the each image; and determining a BCC classification of the input image sequence based on the determined probability of BCC of each image of the input image sequence.

It will be appreciated by a person skilled in the art that other variations and/or modifications may be made to the specific embodiments without departing from the scope of the claimed invention as broadly described. For example, in the description herein, features of different exemplary embodiments may be mixed, combined, interchanged, incorporated, adopted, modified, included etc. or the like across different exemplary embodiments. For example, exemplary embodiments are not necessarily mutually exclusive as some may be combined with one or more embodiments to form new exemplary embodiments. Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments. The present embodiments are, therefore, to be considered in all respects to be illustrative and not restrictive.

1. A system for classification of basal cell carcinoma (BCC), the system comprising,
a processing module configured to receive an input image sequence comprising a plurality of images obtained using a confocal microscopy device; and
a prediction module coupled to the processing module, the prediction module comprising a hierarchical ensemble structure of a plurality of classification models;
wherein the processing module is configured to process each image as a whole image to obtain a plurality of first sub-sections and to further process each first sub-section to obtain a corresponding plurality of second sub-sections;
further wherein the processing module is configured to transmit the each first sub-section to a first classification model of the prediction module trained to process the first sub-sections and to transmit each of the corresponding plurality of second sub-sections to a second classification model of the prediction module trained to process the second sub-sections, the processing module also configured to transmit the whole image to a third classification model of the prediction module trained to process the whole image;
wherein the prediction module is configured to provide a respective block prediction result corresponding to the each first sub-section, the respective block prediction result based on respective prediction results of the first classification model for the each first sub-section and the second classification model for the corresponding plurality of second sub-sections;
wherein the prediction module is configured to provide a preliminary whole image prediction result for the each image based on the third classification model;
further wherein the prediction module is configured to determine a probability of BCC of the each image of the input image sequence based on the preliminary whole image prediction result and a plurality of the respective block prediction results corresponding to the first sub-sections of the each image; and
further wherein the prediction module is configured to determine a BCC classification of the input image sequence based on the determined probability of BCC of each image of the input image sequence.

2. The system as claimed in claim 1, further comprising the prediction module being configured to determine the BCC classification of the input image sequence based on the determined probability of BCC of each image of the input image sequence along a depth of the input image sequence.

3. The system as claimed in claim 1, wherein a graphical representation of the determined probability of BCC of each image of the input image sequence along the depth of the input image sequence is generated.

4. The system as claimed in claim 1, wherein the BCC classification of the input image sequence is determined based on an equation:
$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$
where Sₐᵢ refers to an area of each consecutive part of the graphical representation between a determined probability value of 0.8 to 1, S_{bj} refers to an area of each consecutive part of the graphical representation between a determined probability value of 0.6 to 0.8, S_{ck} refers to an area of each consecutive part of the graphical representation between a determined probability value of 0.4 to 0.6, lₐᵢ refers to a corresponding length of Sₐᵢ in the graphical representation, l_{bj} refers to a corresponding length of S_{bj} in the graphical representation and l_{ck} refers to a corresponding length of S_{ck} in the graphical representation.

5. The system as claimed in claim 1, further comprising the prediction module being configured to provide the respective block prediction result corresponding to the each first sub-section by processing the respective prediction results of the first classification model for the each first sub-section and the second classification model for the corresponding plurality of second sub-sections with a block support vector machine (SVM).

6. The system as claimed in claim 1, further comprising the prediction module being configured to determine the probability of BCC of the each image of the input image sequence by processing the preliminary whole image prediction result and the plurality of the respective block prediction results corresponding to the first sub-sections of the each image with an image-wise stage support vector machine (SVM).

7. The system as claimed in claim 1, further comprising the processing module being configured to perform training of the first classification model, the second classification model and the third classification model using at least two training groups of images;
wherein the at least two training groups of images are used for training the third classification model and are denoted as a whole image dataset;
wherein the system further comprises, based on the whole image dataset, the processing module being configured to process each image of the whole image dataset to obtain a plurality of first training sub-sections and the first training sub-sections are denoted as a first training sub-sections dataset, and wherein the first training sub-sections dataset is used for training the first classification model; and
wherein the system further comprises, based on the first training sub-sections dataset, the processing module being configured to process each first training sub-sections to obtain a plurality of second training sub-sections and the second training sub-sections are denoted as a second training sub-sections dataset, and wherein the second training sub-sections dataset is used for training the second classification model.

8. (canceled)

9. (canceled)

10. The system as claimed in claim 1, further comprising the processing module being configured to generate a visual heatmap based on the respective prediction results of the first classification model for the each first sub-section and the second classification model for the corresponding plurality of second sub-sections, the heatmap being generated by processing the respective prediction results of the first classification model for the each first sub-section and the second classification model for the corresponding plurality of second sub-sections to form a heatmap mask for a combination with the whole image and by providing colour transformation to the combination with the whole image.

11. (canceled)

12. (canceled)

13. (canceled)

14. (canceled)

15. A method of classifying basal cell carcinoma (BCC), the method comprising,
providing a processing module;
inputting an input image sequence comprising a plurality of images obtained using a confocal microscopy device to the processing module;
coupling a prediction module to the processing module, the prediction module comprising a hierarchical ensemble structure of a plurality of classification models;
processing each image, by the processing module, as a whole image to obtain a plurality of first sub-sections and further processing, by the processing module, each first sub-section to obtain a corresponding plurality of second sub-sections;
transmitting, by the processing module, the each first sub-section to a first classification model of the prediction module trained to process the first sub-sections;
transmitting, by the processing module, each of the corresponding plurality of second sub-sections to a second classification model of the prediction module trained to process the second sub-sections;
transmitting, by the processing module, the whole image to a third classification model of the prediction module trained to process the whole image;
providing, by the prediction module, a respective block prediction result corresponding to the each first sub-section, the respective block prediction result based on respective prediction results of the first classification model for the each first sub-section and the second classification model for the corresponding plurality of second sub-sections;
providing, by the prediction module, a preliminary whole image prediction result for the each image based on the third classification model;
determining, by the prediction module, a probability of BCC of the each image of the input image sequence based on the preliminary whole image prediction result and a plurality of the respective block prediction results corresponding to the first sub-sections of the each image; and
determining, by the prediction module, a BCC classification of the input image sequence based on the determined probability of BCC of each image of the input image sequence.

16. The method as claimed in claim 15, further comprising determining, by the prediction module, the BCC classification of the input image sequence based on the determined probability of BCC of each image of the input image sequence along a depth of the input image sequence.

17. The method as claimed in claim 15, further comprising generating a graphical representation of the determined probability of BCC of each image of the input image sequence along the depth of the input image sequence.

18. The method as claimed in claim 15, wherein the BCC classification of the input image sequence is determined based on an equation:
$$B = ln ( {\sum }_{i} {S}_{ai} {e}^{2 {l}_{a i}} + {\sum }_{j} {S}_{b j} {e}^{{l}_{b j}} + {\sum }_{k} {S}_{c k} {l}_{c k} )$$
where Sₐᵢ refers to an area of each consecutive part of the graphical representation between a determined probability value of 0.8 to 1, S_{bj} refers to an area of each consecutive part of the graphical representation between a determined probability value of 0.6 to 0.8, S_{ck} refers to an area of each consecutive part of the graphical representation between a determined probability value of 0.4 to 0.6, lₐᵢ refers to a corresponding length of Sₐᵢ in the graphical representation, l_{bj} refers to a corresponding length of S_{bj} in the graphical representation and l_{ck} refers to a corresponding length of S_{ck} in the graphical representation.

19. The method as claimed in claim 15, further comprising providing, by the prediction module, the respective block prediction result corresponding to the each first sub-section by processing the respective prediction results of the first classification model for the each first sub-section and the second classification model for the corresponding plurality of second sub-sections with using a block support vector machine (SVM).

20. The method as claimed in claim 15, further comprising determining, by the prediction module, the probability of BCC of the each image of the input image sequence by processing the preliminary whole image prediction result and the plurality of the respective block prediction results corresponding to the first sub-sections of the each image with using an image-wise stage support vector machine (SVM).

21. The method as claimed in claim 15, further comprising performing, by the processing module, training of the first classification model, the second classification model and the third classification model using at least two training groups of images;
wherein the at least two training groups of images are used for training the third classification model and are denoted as a whole image dataset;
wherein the method further comprises, based on the whole image dataset, processing, by the processing module, each image of the whole image dataset to obtain a plurality of first training sub-sections and the first training sub-sections are denoted as a first training sub-sections dataset, and training the first classification model with the first training sub-sections dataset; and
wherein the method further comprises, based on the first training sub-sections dataset, processing, by the processing module, each first training sub-sections to obtain a plurality of second training sub-sections and the second training sub-sections are denoted as a second training sub-sections dataset, and training the second classification model with the second training sub-sections dataset.

22. (canceled)

23. (canceled)

24. The method as claimed in claim 15, further comprising forming a heatmap mask by processing the respective prediction results of the first classification model for the each first sub-section and the second classification model for the corresponding plurality of second sub-sections; combining the heatmap mask with the whole image; and providing colour transformation to the combination with the whole image to generate a visual heatmap.

25. (canceled)

26. (canceled)

27. (canceled)

28. (canceled)

29. (canceled)
